(19) United States
(12) Patent Application Publication
(10) Pub. No.: US 2009/0192191 A1
(43) Pub. Date: Jul. 30, 2009

(54) Substituted hydroxamic acid derivatives as tnf inhibitors

(76) Inventors: Braj Bhushan Lohray, Gujarat (IN); Vidya Bhushan Lohray, Gujarat (IN); Mukul R. Jain, Gujarat (IN); Pravin S. Thombare, Gujarat (IN)

Correspondence Address: NIXON & VANDERHYE, PC, 901 NORTH GLEBE ROAD, 11TH FLOOR, ARLINGTON, VA, 22203, US

(21) Appl. No.: 10/585,420

(22) PCT Filed: Jan. 7, 2005
(86) PCT No.: PCT/IN2005/000010

§ 371 Date: Aug. 28, 2006

(30) Foreign Application Priority Data
Jan. 9, 2004 (IN) 22/MUM/2004

(51) Int. Cl. A61K 31/4709; C07D 401/12; C07D 207/38; C07D 405/14; C07D 409/14; A61K 31/4015; A61P 19/02

(52) U.S. Cl. 514/314; 546/173; 548/550; 514/424

(57) ABSTRACT

The present invention relates to novel compounds of formula (I) and their pharmaceutically useful compositions as MMP and TNF inhibitors.

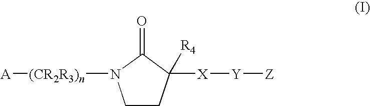

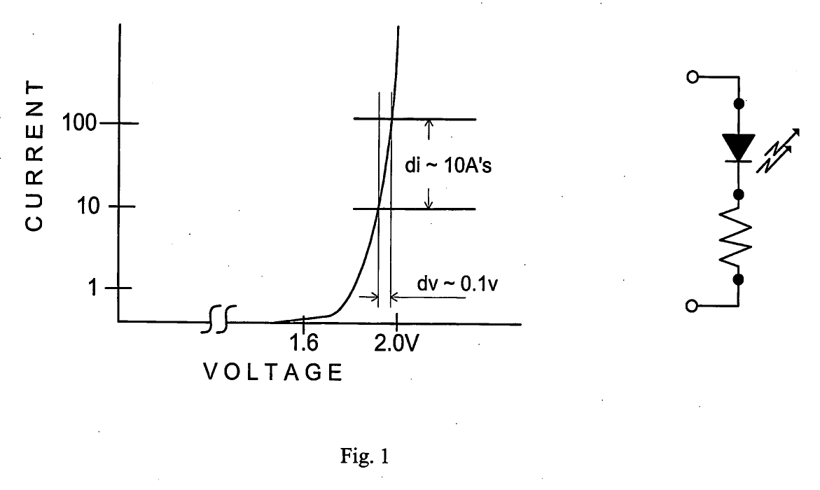

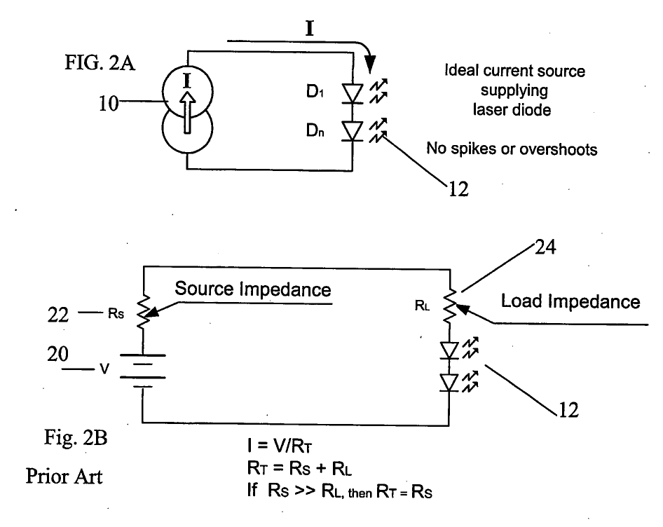

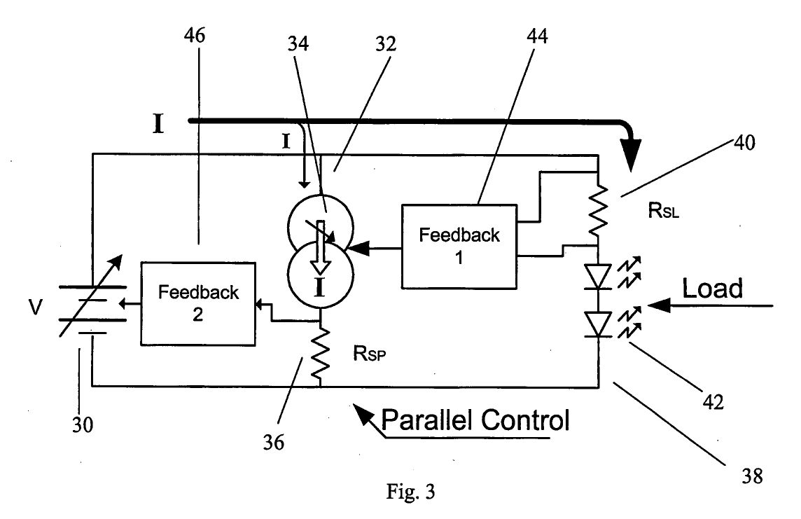

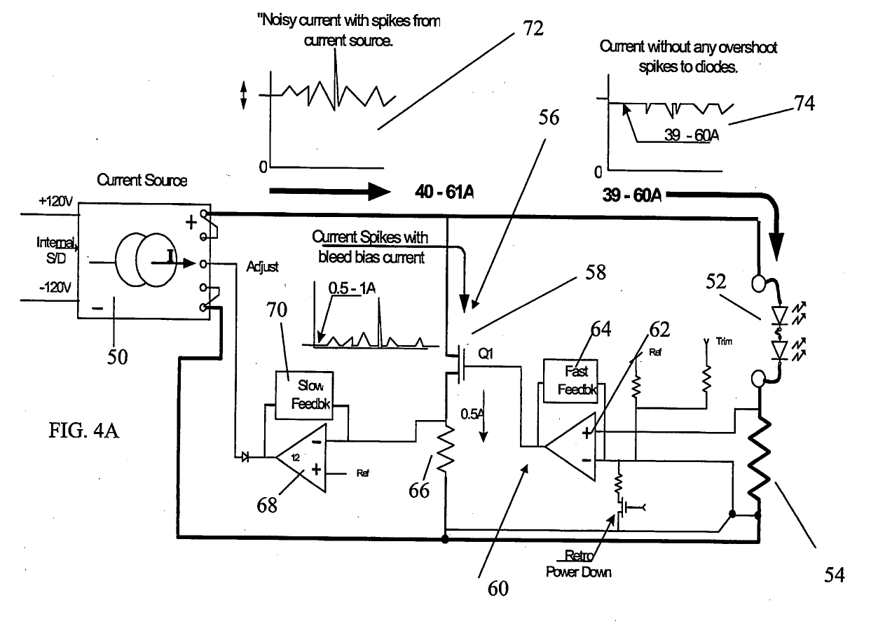

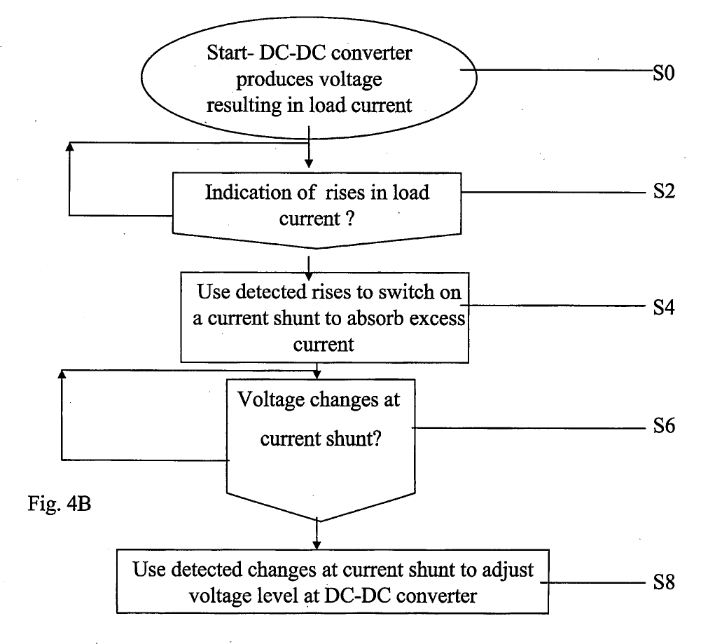

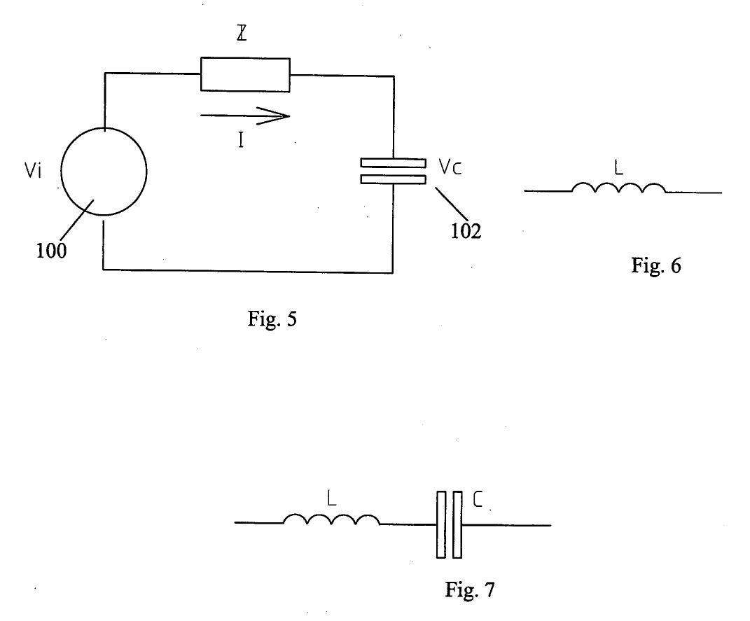

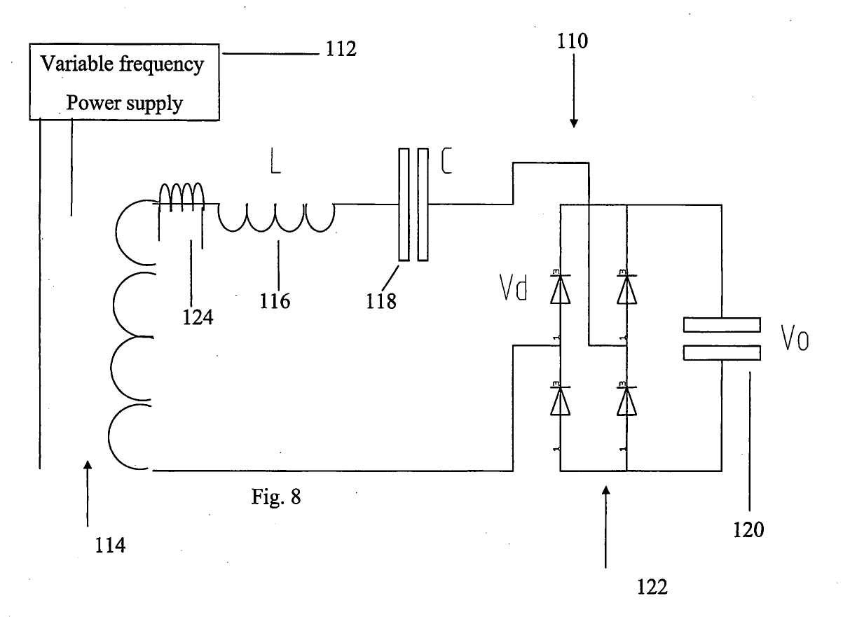

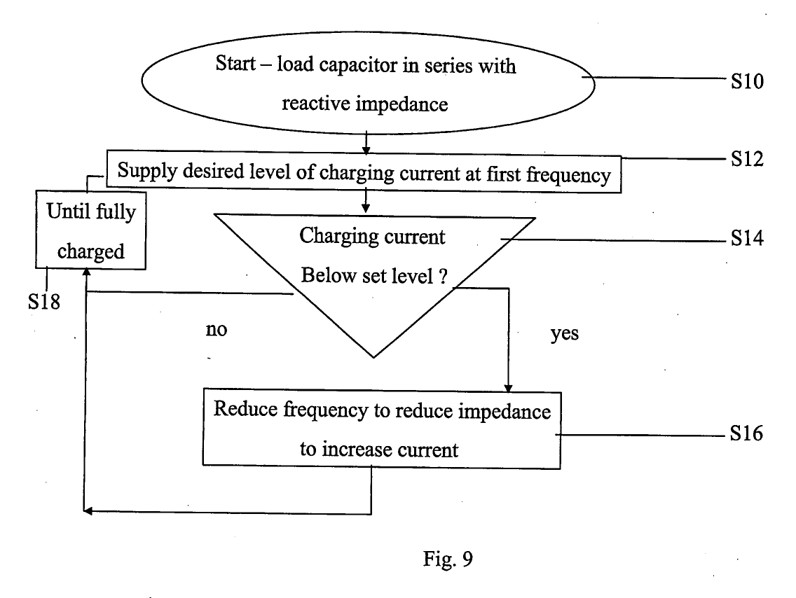

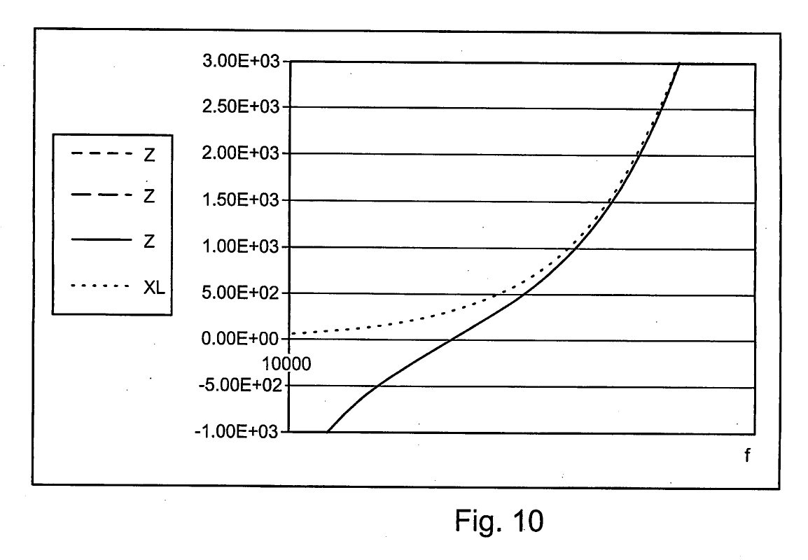

### FIELD OF INVENTION

The present invention relates to novel compounds having MMP and TNF inhibitory activities, their stereoisomers, their tautomeric forms, their pharmaceutically acceptable salts, their pharmaceutically acceptable solvates and pharmaceutically acceptable compositions containing them. More particularly, the present invention relates to novel hydroxamic acids of the general formula (I), their stercoisomers, their tautomeric forms, their pharmaceutically acceptable salts, their pharmaceutically acceptable solvates, pharmaceutical compositions containing them, use of these compounds in medicine and the intermediates involved in their preparation.

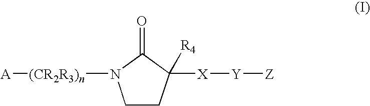

The present invention also relates to a process for the preparation of the above said novel compounds, their tautomeric forms, their stereoisomers, their pharmaceutically acceptable salts, their pharmaceutically acceptable solvates, and pharmaceutical compositions containing them.

The compounds of the present invention are useful as inhibitors of matrix-degrading metalloproteinases, reprolysin (also known as adamylsin) subfamilies and of INF-alpha (tumor necrosis factor alpha) activity. The invention also describes a method of inhibiting TNF-alpha and matrix degrading metalloproteinase activity and a method of treating TNF-alpha and matrix metalloproteiase dependent diseases or conditions in mammals which are responsive to matrix metalloprotease and TNF-alpha inhibition, using such compounds of this invention or pharmaceutical compositions comprising such compounds of this invention.

### BACKGROUND OF INVENTION

A number of enzymes effect the breakdown of structural proteins and many of them are structurally related to metalloproteases especially of the zinc metalloendopeptidases family. Matrix-degrading metalloproteinases (MMP) and reprolysin are examples of zinc metalloendopeptidases. Matrix-degrading metalloproteinases (MMP), such as gelatinase, stromelysin and collagenase, have been found to play an important role in the uncontrolled breakdown of connective tissue, including proteoglycan and collagen, which leads to resorption of the extracellular matrix. This is a feature of many pathological conditions, such as artritis (e.g. osteoarthritis and rheumatoid arthritis), tissue ulceration (e.g. corneal, epidermal and gastric ulceration), abnormal wound healing, periodontal disease, bone diseases (e.g. osteoporosis and Paget's disease), tumor metastasis or invasion, as well as HIV-infection.

The mammalian reprolysins are known as ADAMs (A Disintegrin And Metalloproteinase) [Wolfberg, et. al., J Cell Biol., 131, 275-78 (1985)] and they contain a disintegrin domain in addition to a metalloproteinase-like domain. Of the twenty-three different ADAMs identified, ADAM-17, also known as tumor necrosis factor-alpha converting enzyme (TACE) is the most well known. TACE is responsible for cleavage of cell bound tumor necrosis factor-alpha (TNF-α, also known as cachectin). TNF-α is recognized to be involved in many infectious and auto-immune diseases [W. Friers, FBS Letters, 285, 199 (1991)]. Further, TNF-α has been shown to be the prime mediator of the inflammatory response seen in sepsis and septic shock. Two forms of TNF-α are known, a type II membrane protein having a relative molecular mass of 26 kD and a soluble 17 kD form, which is generated from the cell bound protein by specific proteolytic cleavage. The 17 kD TNF-α is released by the cell and is associated with the deleterious effects of TNF-α. Another feature of this form of TNF-α is that it can act at sites remote from the site of synthesis. Compounds, which are inhibitors of TACE, prevent the formation of TNF-α and prevent the deleterious effects of the soluble factor.

The compounds of the invention are useful in the treatment of but not limited to arthritis (including osteoarthritis and rheumatoid arthritis), inflammatory bowel disease, Crohn's disease, emphysema, malaria, acute respiratory distress syndrome, asthma, chronic obstructive pulmonary disease, Alzheimer's disease, organ transplant toxicity, cachexia, allergic reactions, inflammation, allergic contact hypersensitivity, cancer (such as solid tumor cancer including colon cancer, breast cancer, lung cancer and prostrate cancer and hematopoletic malignancies including leukemias and lymphomas), mycobacterial infection, meningitis, graft rejection, tissue ulceration, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), congestive heart failure, myocardial infarction, stroke, cerebral ischemia, head trauma, spinal cord injury, neuro-degenerative disorders (acute and chronic), autoimmune disorders, Huntington's disease, Parkinson's disease, migraine, depression, hyperoxic alveolar injury, peripheral neuropathy, pain, cerebral amyloid angiopathy, nootropic or cognition enhancement, amyotrophic laeral sclerosis, multiple sclerosis, ocular angiogenesis, corneal injury, macular degeneration, abnormal wound healing, burns, diabetes, tumor invasion tumor growth, tumor metastasis, corneal scarring, scleritis, AIDS, sepsis or septic shock.

Since excessive TNF production has been noted in several disease conditions also characterized by MMP-mediated tissue degradation, compounds which inhibit both MMPs and TNF-A production may also have a particular advantage in diseases where both mechanisms are involved.

The compounds which inhibits TACE activity are described in WO 2004002956, WO 03082287, WO0228846, WO0204416, WO0170673, WO0059285, WO0059874, WO0044710, WO9965867, WO 9958531, US 2003/0225054, U.S. Pat. No. 6,620,823, U.S. Pat. No. 6,268,379, U.S. Pat. No. 6,153,757, U.S. Pat. No. 6,057,336, U.S. Pat. No. 6,114,361, EP1134215, EP1041072 which are all incorporated herein as reference.

U.S. Pat. No. 6,057,336 describes MMP inhibitors of formula (A):

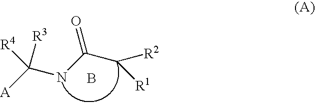

wherein
A is selected from —CONHOH, —CONHOR⁵, —COR⁵, —CO₂H, —CH₂CO₂H, —CO₂R⁶, —N(OH)COR⁵, —SH, —CH₂SH, —SO₂NHR^{a}, —SN₂H₂R^{a}, —PO(OH)₂, and —PO(OH)NHR^{a}; ring B is a 4-8 membered cyclic amide containing from 0-3 additional heteroatonms selected from O, NR^{a}, and S(O)ₚ, 0-1 additional carbonyl groups and 0-1 double bonds; R¹ is U-X-Y-Z-U^{a}-X^{a}-Y^{a}-Z^{a};
U is absent or selected from: O, —NR, C(O), C(O)O, OC(O), CONR^{a}, NR^{a}C(O), OC(O)O, OC(O)NR^{a}, NR^{a}C(O)O, NR^{a}C(O)NR^{a}, S(O)ₚ, S(O)ₚNR^{a}, NR^{a}S(O)ₚ, and NR^{a}SO₂NR^{a};
X is absent or selected from (C₁₋₁₀)alkylene, (C₂₋₁₀)alkenylene, and (C₂₋₁₀) alkynylene;
Y is absent or selected from O, NR^{a}, S(O)ₚ, and C(O);
Z is absent or selected from a (C₃₋₁₃)carbocyclic residue which may be substituted with 0-5 R^{b} and a 5-14 membered heterocyclic system containing 1-4 heteroatoms selected from the group consisting of N, O, and S and substituted with 0-5 R^{b};
U^{a} is absent or is selected from: O, NR^{a}, C(O), C(O)O, OC(O), CONR^{a}, NR^{a}C(O), OC(O)O, OC(O)NR^{a}, NR^{a}C(O)O, NR^{a}C(O)NR^{a}, S(O)ₚ, S(O)ₚNR^{a}, NR^{a}S(O)ₚ, and NR^{a}SO₂NR^{a}; X^{a} is absent or selected from (C₁₋₁₀)alcylene, (C₂₋₁₀)alkenylene, and (C₂₋₁₀)alkylene; Y^{a} is absent or selected from O, NR^{a}, S(O)ₚ, and C(O); Z^{a} is absent or selected from a (C₃₋₁₃) carbocyclic residue which may be substituted with 0-5 R^{c} and a 5-14 membered heterocyclic system containing 1-4 heteroatoms selected from the group consisting of N, O, and S and substituted with 0-5 R^{c}; R^{b}, at each occurrence, is independently selected from (C₁₋₆)alkyl, OR^{a}, Cl, F, Br, I, ═O, CN, NO2, NR^{a}R^{a′}, C(O)R^{a}, C(O)OR^{a}, C(O)NR^{a}R^{a′}, S(O)₂NR^{a}R^{a′}, S(O)ₚR^{a}, CF₃ and CF₂CF₃; R^{c} at each occurrence, is independently selected from (C₁₋₆)alkyl, OR^{a}, Cl, F, Br, I, ═O, CN, NO₂, NR^{a}R^{a′}, C(O)R^{a}, C(O)OR^{a}, C(O)NR^{a}R^{a}, S(O)₂NR^{a}R^{a′}, S(O)ₚR^{a}, CF₃ and CF₂CF₃, —CH(═NOH), —(═NOH)CH₃, (CRR′)ₛO(CRR′)_{s′}R^{d}, (CRR′)ₛS(O)ₚ(CRR′)_{s′}R^{d}, (CRR′)ₛNR^{s}(CRR′)_{s′}R^{d}, phenyl, and a 5-14 membered heterocyclic system containing from 1-4 heteroatoms selected from the group consisting of N, O and S;
R⁵ at each occurrence, is selected from (C₁₋₁₀)alkyl substituted with 0-2 R^{b}, and (C₁₋₈) alkyl substituted with 0-2 R^{d}; R^{d}, at each occurrence is independently selected from phenyl substituted with 0-3 R^{b}, biphenyl substituted with 0-2 R^{b}.
US 2003/0225054 also discloses TACE inhibitors having a similar general formula as U.S. Pat. No. 6,057,336. In this application, R^{c} denotes a (C₁₋₆)alkyl, OR^{a}, Cl, F, Br, I, ═O, CN, NO₂, NR^{a}R^{a′}, C(O)R^{a}, C(O)OR^{a}, C(O)NR^{a}R^{a′}, S(O)₂NR^{a}R^{a′}, S(O)ₚR^{a}, CF₃ and CF₂CF₃, —CH(═NOH), —(=NOH)CH₃, (CRR′)ₛO(CRR′)_{s′}R^{d}, (CRR′)ₛS(O)ₚ(CRR′)_{s′}R^{d}, (CRR′)ₛNR^{a}(CRR′)_{s′}R^{d}, phenyl, (CH₂)ᵣ-(C₃₋₆) membered carbocyle and a (CH₂)ᵣ-C₅₋₆) membered heterocyclic system containing from 1-4 heteroatoms selected from the group consisting of N, O and S.

### SUMMARY OF INVENTION

The present invention relates to novel compounds of formula (I) having MP and TNF inhibitory activities. Such compounds are useful in the treatment of diseases such as arthritis (e.g. osteoarthritis and rheumatoid arthritis), tissue ulceration (e.g. corneal, epidermal and gastric ulceration), abnormal wound healing, periodontal disease, bone diseases (e.g. osteoporosis and Paget's disease), tumor metastasis or invasion.

### OBJECTS OF THE INVENTION

The main object of the present invention is to provide novel substituted hydroxamic acid derivatives represented by the general formula (I), their stereoisomers, their tautomeric forms, their pharmaceutically acceptable salts, their pharmaceutically acceptable solvates, and pharmaceutical compositions containing them or their mixtures thereof.

Another object of the present invention is to provide novel substituted hydroxamic acid derivatives represented by the general formula (I), their stereoisomers, their tautomeric forms, their pharmaceutically acceptable salts, their pharmaceutically acceptable solvates, and pharmaceutical compositions containing them or their mixtures thereof having enhanced activities, without toxic effects or with reduced toxic effect.

Yet another object of this invention is to provide a process for the preparation of novel substituted hydroxamic acid derivatives represented by the general formula (I), their stereoisomers, their tautomeric forms, their stereoisomers, their pharmaceutically acceptable salts, their pharmaceutically acceptable solvates.

Still another object of the present invention is to provide pharmaceutical compositions containing compounds of the general formula (I), their tautomeric forms, their stereoisomers, their pharmaceutically acceptable salts, their pharmaceutically acceptable solvates or their mixtures in combination with suitable carriers, solvents, diluents and other media normally employed in preparing such compositions.

A further object of the present invention is to provide process for preparation of intermediates involved in the process.

### DETAILED DESCRIPTION OF THE INVENTION

Accordingly, the present invention relates to compounds of the general formula (I),

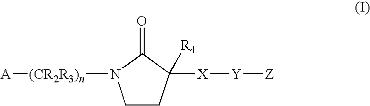

their stereoisomers, their tautomeric forms, their pharmaceutically acceptable salts, their pharmaceutically acceptable solvates, wherein

A is selected from COR₁, CO₂H, CH₂CO₂H, CONHOH, CONHOR₁, N(OH)COR₁, C(═NOR₁)NHR₁, SH, CH₂SH, SO₂NHR₁ & S(═NH)₂R₁.

R₁ represents hydrogen, substituted or unsubstituted groups selected from linear or branched (C₁-C₈)alkyl, (C₃-C₇)cycloalkyl, acyl, aryl, aralkyl, alkylaminocarbonyl, arylaminocarbonyl, aralkylaminocarbonyl, heteroarylaminocarbonyl, heteroaralkylaminocarbonyl, or heterocyclylaminocarbonyl;
R₂ and R₃ may be same or different and independently represent hydrogen, halogen, substituted or unsubstituted groups selected from linear or branched (C₁-C₈)alkyl, (C₃-C₇)cycloalkyl, acyl, groups, substituted or unsubstituted groups selected from (C₃-C₇)cycloallyl, aryl, aralkyl, heteroaryl, heterocycle groups;
X represents optionally substituted (C_{3-C}₁₃) carbocyclic residue or a 5-14 membered heterocyclic system containing 1-4 heteroatoms selected from the group consisting of N, O, or S,
Z represents substituted (C₃-C₁₃) carbocyclic residue or a 5-14 membered substituted heterocyclic system containing 1-4 heteroatoms selected from the group consisting of N, O, or S; preferably Z represents heterocyclic group such as pyrrolidinyl, imidazolidinyl, piperidinyl, piperazinyl dihydrothiophene, dihydropyran, dihydrofrun, dihydrothiazole, pyridyl, thienyl, furyl, pyrrolyl oxazlyl, thiazolyl, isothiazolyl, imidazolyl, isoxazolyl, oxadiazolyl, thiadiaolyl, triazolyl, tetrazolyl, benzopyranyl, benzopyranonyl, benzofuranyl, benzothienyl, indolinyl, indolyl, azaindolyl, azaindolinyl, benzodihydrofuranyl, benzodihydrothienyl, pyrazolopyrimidinyl, pyrazolopyrimidonyl, azaquinazolinyl, azaquinazolinoyl, pyridofuranyl, pyridothienyl, thienopyrimidyl, thienopyrimidonyl, quinolinyl, pyrimidinyl, pyrazolyl, quinazolinyl, quinazolonyl, pyrimidonyl, pyridazinyl, triazinyl, benzoxazinyl, benzoxazinonyl, benzothiazinyl benzothiazinonyl, benzoxazolyl, benzothiazolyl, benzimidazolyl, benzotriazolyl and the like; more preferably Z represents quinolinyl, pyrimidinyl, quinazolinyl groups;
The substituents on Z may be selected optionally substituted alkenyl, optionally substituted alkynyl, optionally substituted alkoxy alkyl or substituted groups selected from (CH₂)ᵣ-(C₃₋₆)cycloalkyl, (CH₂)ᵣ-cycloalkenyl, (CH₂)ᵣ-phenyl, or (CH₂)ᵣ-(3-14) membered heterocycle comprising 1-4 heteroatoms selected from the group consisting N, O and S; n=1-2; r=0-6;
Y represents (CR′R″)ₚ, O(CR′R″)ₚ, (CR′R″)ₚO, C(O)(CR′R″)ₚ, (CR′R″)C(O), NR′(CR′R″)ₚ, NR′NR″, (CR′R″NR″, NR′C(O)(CR′R″)ₚ, CONR′(CR′R″)ₚ, (CR′R″)ₚNR′C(O), (CR′R″)ₚNR′C(O), (CR′R″)ₚC(O)NR′, NR′CONR′, (CR′R″)ₚS(O)_{q}, S(O)_{q}(CR′R″)ₚ, wherein p=0-2 and q=0-2; R′ and R″ may be same or different and independently represent H, alkyl group, linear or branched substituted or unsubstituted (C₁-C₆)alkyl, linear or branched substituted or unsubstituted (C₁-C₆)alkenyl, linear or branched substituted or unsubstituted (C₁-C₆)alkynyl groups;
R₄ represents H, SR′, halogen, NR′R″, OR′, CN, NO₂, (C₁-CO₁₀)alkyl-R^{a}, (C₂-C₁₀)alkenyl-R^{a}, (C₂-C₁₀)alkynyl-R^{a}, (CR′R″)ₚ-R^{a}, O(CR′R″)ₚR^{a}, (CR′R″)ₚO(CR′R″)ₚR^{a}, (CR′R″)ₚNR′(CR′R″)ₚR^{a}, (CR′R″)ₚC(O)(CR′R″)ₚR^{a}, (CR′R″)ₚOC(O)(CR′R″)ₚR^{a}, (CR′R″)ₚC(O)O(CR′R″)ₚR^{a}, (CR′R″)ₚNR′C(O)(CR′R″)ₚR^{a}, (CR′R″)ₚC(O)NR′(CR′R″)ₚR^{a}, (CR′R″)ₚS(O)_{q}(CR′R″)ₚR^{a}, (CR′R″)ₚS(O)_{q}NR′(CR′R″)ₚR^{a}, (CR′R″)ₚNR′S(O)_{q}(CR′R″)ₚR^{a}, (CR′R″)ₚOC(O)NR′(CW′R″)ₚR^{a}, (CR′R″)ₚNR′C(O)O(CR′R″)ₚR^{a}, wherein p=0-2 and q=0-2; R′ and R″ may be same or different and independently represent H, alkyl group, linear or branched substituted or unsubstituted (C₁-C₆)alkyl, linear or branched substituted or unsubstituted (C₁-C₆)alkenyl, linear or branched substituted or unsubstituted (C₁-C₆)alkynyl groups, where R^{a} may represent H, halogen, alkyl group, linear or branched substituted or unsubstituted (C₁-C₆)alkyl, linear or branched substituted or unsubstituted (C₁-C₆)alkenyl, linear or branched substituted or unsubstituted (C₁-C₆)alknyl groups;

The term “substituted” used alone or in combination with other radicals, denotes suitable substituents on that radical such as substituted alkyl, substituted alkenyl substituted alkynyl substituted cycloalkyl, substituted aryl, etc, mentioned anywhere in the specification. The suitable substituents include, but are not limited to the following radicals, alone or in combination with other radicals, such as, hydroxyl, oxo, halo, thio, nitro, amino, cyano, formyl, alkyl, haloalkyl, perhaloalkyl, alkoxy, haloalkoxy, perhaloalkoxy, alkenyl, alkynyl, cycloalkyl, cycloalkenyl bicycloalkyl, bicycloalkenyl, alkoxy, alkenoxy, cycloalkoxy, aryl, aryloxy, aralkyl, aralkoxy, heterocyclyl, heteroaryl, heterocycloalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, aminocarbonylamino, alkylaminocarbonylamino, alkoxyamino, hydroxyl amino, sulfonyloxy, alkylsulfonyloxy, alkoxycarbonylamino, aryloxycarbonylamino, aralkyloxycarbonylanino sulfenyl derivatives, sulfonyl derivatives, sulfonic acid and its derivatives.

The substituents on any of the substitutions, if substituted, may be selected from hydroxyl, oxo, halo, thio, nitro, amino, cyano, formyl, or substituted or unsubstituted groups selected from alkyl, haloalkyl, perhaloalkyl, alkoxy, haloalkoxy, perhaloalkoxy, alkenyl, alkynyl cycloalkyl, cycloalkenyl, bicycloalkyl, bicycloalkenyl, alkoxy, alkenoxy, cycloalkoxy, aryl, aryloxy, aralkyl, aralkoxy, heterocylyl; heteroaryl, heterocyclylalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, alkylsulfonyloxy, alkoxycarbonylamino, aryloxycarbonylamino, aralkyloxycarbonylamino, aminocarbonylamino, alkylaminocarbonylamino, alkoxyamino, hydroxyl amino, sulfenyl derivatives, sulfonyl derivatives, sulfonic acid and its derivatives.

The various groups, radicals and substituents used anywhere in the specification are described in the following paragraphs.

The term “alkyl” used herein, either alone or in combination with other radicals, denotes a linear or branched radical containing one to twelve carbons, such as methyl, ethyl, n-propyl, iso-propyl, n-butyl, sec-butyl, tert-butyl, amyl t-amyl n-pentyl n-hexyl, iso-hexyl, heptyl, octyl and the like.

The term “alkenyl” used herein, either alone or in combination with other radicals, denotes a linear or branched radical containing two to twelve carbons such as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4heptenyl, 5-heptenyl, 6-heptenyl and the like. The term “alkenyl” includes dienes and trienes of straight and branched chains.

The term “alkynyl” used herein, either alone or in combination with other radicals, denotes a linear or branched radical containing two to twelve carbons, such as ethynyl, 1-propynyl, 2-propynyl, 1-butynyl, 2-butynyl, 3-butynyl, 1-pentynyl 2-pentynyl, 3-pentynyl, 4-pentynyl, 1-hexynyl, 3-hexynyl, 4-hexynyl, 5-hexynyl, and the like. The term “alkynyl” includes di- and tri-ynes.

The term “cycloalkyl” used herein, either alone or in combination with other radicals, denotes a radical containing three to seven carbons, such as cyclopropyl, cyclobutyl, cyclopentyl, cyclohexyl, cycloheptyl and the like.

The term “cycloalkenyl” or used herein, either alone or in combination with other radicals, denotes a radical containing three to seven carbons, such as cyclopropenyl 1-cyclobutenyl, 2-cylobutenyl, 1-cyclopentenyl, 2-cyclopentenyl, 3-cyclopentenyl, 1-cyclohexenyl, 2-cyclohexenyl, 3-cyclohexenyl, 1-cycloheptenyl, cycloheptadienyl, cycloheptatrienyl, and the like.

The term “carbocycle” or “carbocyclic” used herein, either alone or in combination with other radicals, denotes any stable (C₃-C₇) membered monocyclic or bicyclic, or (C₇-C₁₃) membered bicyclic or tricyclic, any of which may be saturated, partially unsaturated, or aromatic. Examples of such carbocycles includes but are not limited to cyclopropyl, cyclobutyl, cyclopentyl, cyclopropenyl, 1-cyclobutenyl, 2-cylobutenyl, 1-cyclopentenyl, 2-cyclopentenyl, 3-cyclopentenyl 1-cyclohexenyl, [3,3,0]bicyclooctayl [4,3,0]bicyclononyl, [4,4,0]bicyclodecanyl, [2,2,2]bicyclooctanyl, fluorenyl, phenyl, napthyl, indanyl, adamantyl, and tetrahydronaphthyl and the like.

The term “alkoxy” used herein, either alone or in combination with other radicals, denotes an alkyl radical, as defined above, attached directly to an oxygen atom, such as methoxy, ethoxy, n-propoxy, iso-propoxy, n-butoxy, t-butoxy, iso-butoxy, pentyloxy, hexyloxy, and the like.

The term “alkenoxy” used herein, either alone or in combination with other radicals, denotes an alkenyl radical, as defined above, attached to an oxygen atom, such as vinyloxy, allyloxy, butenoxy, pentenoxy, hexenoxy, and the like.

The term “cycloalkoxy” used herein, either alone or in combination with other radicals, denotes a cycloalkyl radical as defined above, attached directly to an oxygen atom, such as cyclopropyloxy, cyclobutyloxy, cyclopentyloxy, cyclohexyloxy, cycloheptyloxy and the like.

The term “halo” or “halogen” used herein, either alone or in combination with other radicals, such as “haloalkyl”, “perhaloalkyl” etc refers to a fluoro, chloro, bromo or iodo group. The term “aloalkyl” denotes a alkyl radical, as defined above, substituted with one or more halogens; such as perhaloalkyl, more preferably, perfluoro(C₁-C₆)alkyl such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups. The term “haloalkoxy” denotes a haloalkyl, as defined above, directly attached to an oxygen atom, such as fluoromethoxy, chloromethoxy, fluoroethoxy chloroethoxy groups, and the like. The term “perhaloalkoxy” denotes a perhaloalkyl radical, as defined above, directly attached to an oxygen atom, trifluoromethoxy, trifluoroethoxy, and the like.

The term “aryl” or “aromatic” used herein, either alone or in combination with other radicals, denotes an aromatic system containing one, two or three rings wherein such rings may be attached together in a pendant manner or may be fused, such as phenyl, naphthyl, tetrahydronaphthyl, indane, biphenyl, and the like. The term “aralkyl” denotes an alkyl group, as defined above, attached to an aryl, such as benzyl, phenethyl, naphthylmethyl, and the like. The term “aryloxy” denotes an aryl radical, as defined above, attached to an alkoxy group, such as phenoxy, naphthyloxy and the like, which may be substituted. The term “aralkoxy” denotes an arylalkyl moiety, as defined above, such as benzyloxy, phenethyloxy, naphthylmethyloxy, phenylpropyloxy, and the like, which may be substituted.

The term “heterocyclyl” or “heterocyclic” used herein, either alone or in combination with other radicals, denotes saturated, partially saturated, unsaturated or aromatic ring-shaped radicals, the heteroatoms selected from nitrogen, sulfur and oxygen. Examples of saturated heterocyclic radicals include aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, and the like; examples of partially saturated heterocyclic radicals include dihydrothiophene, dihydropyran, dihydrofuran, dihydrothiazole, pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzopyranyl, benzopyranonyl, benzofuranyl, benzothienyl, indolinyl, indolyl, azaindolyl, azaindolinyl, benzodihydrofuranyl, benzodihydrothienyl, pyrazolopyrimidinyl, pyrazolopyrimidonyl, azaquinazolinyl, azaquinazolinoyl, pyridofuranyl, pyridothienyl, thienopyrimidyl, thienopyrimidonyl, quinolinyl, pyrimidinyl, pyrazolyl, quinazolinyl, quinazolonyl, pyrimidonyl, pyridazinyl, triazinyl, benzoxazinyl, benzoxazinonyl, benzothiazinyl, benzothiazinonyl, benzoxazolyl, benzothiazolyl, benzimidazolyl, benzotriazolyl, phthalazynil, naphthylidinyl, purinyl, carbazolyl, phenothiazinyl, phenoxazinyl, and the like.

The term “heterocyclylalkyl” used herein, either alone or in combination with other radicals, represents a heterocyclyl group, as defined above, substituted with an alkyl group of one to twelve carbons, such as pyrrolidinealkyl, piperidinealkyl, morpholinealkyl, thiomorpholinealkyl, oxazolinealkyl, and the like, which may be substituted. The term “heteroaralkyl” used herein, either alone or in combination with other radicals, denotes a heteroaryl group, as defined above, attached to a straight or branched saturated carbon chain containing 1 to 6 carbons, such as (2-furyl)methyl, (3-furyl)methyl, (2-thienyl)methyl, (3-thienyl)methyl, (2-pyridyl)methyl, 1-methyl-1-(2-pyrimidyl)ethyl and the like. The terms “heteroaryloxy”, “heteroaralkoxy”, “heterocycloxy”, “heterocylylalkoxy” denotes heteroaryl, heteroarylalkyl, heterocyclyl, heterocylylalkyl groups respectively, as defined above, attached to an oxygen atom.

The term “acyl” used herein, either alone or in combination with other radicals, denotes a radical containing one to eight carbons such as formyl, acetyl, propanoyl, butanoyl, iso-butanoyl, pentanoyl, hexanoyl, heptanoyl, benzoyl and the like, which maybe substituted.

The term “acyloxy” used herein, either alone or in combination with other radicals, denotes a radical acyl, as defined above, directly attached to an oxygen atom, such as acetyloxy, propionyloxy, butanoyloxy, iso-butanoyloxy, benzoyloxy and the like.

The term “acylamino” used herein, either alone or in combination with other radicals, denotes an acyl group as defined earlier, may be CH₃CONH, C₂H₅CONH, C₃H₇CONH, C₄H₉CONH, C6H₅CONH and the like, which may be substituted.

The term “mono-substituted amino” used herein, either alone or in combination with other radicals, denotes an amino group, substituted with one group selected from (C₁-C₆)alkyl, substituted alkyl, aryl, substituted aryl or arlylalkyl groups. Examples of monoalkylamino group include methylamine, ethylamine, n-propylamine, n-butylamine, n-pentylamine and the like.

The term “disubstituted amino” used herein, either alone or in combination with other radicals, denotes an amino group, substituted with two radicals that may be same or different selected from (C₁-C₆)alkyl, substituted alkyl, aryl, substituted aryl, OT arylalkyl groups, such as dimethylamino, methylethylmino, diethylamino, phenylmethyl amino and the like.

The term “arylamino” used herein, either alone or in combination with other radicals, denotes an aryl group, as defined above, linked through amino having a free valence bond from the nitrogen atom, such as phenylamino, naphthylamino, N-methyl anilino and the like.

The term “aralkylamino” used herein, either alone or in combination with other radicals, denotes an arylalkyl group as defined above linked through amino having a free valence bond from the nitrogen atom e.g. benzylamino, phenethylamino, 3-phenylpropylamino, 1-napthylmethylamino, 21-napthyl)ethylamino and the like.

The term “oxo” or “carbonyl” used herein, either alone (—C═O—) or in combination with other radicals, such as “alkylcarbonyl”, denotes a carbonyl radical (—C═O—) substituted with an alkyl radical such as acyl or alkanoyl, as described above.

The term “carboxylic acid” used herein, alone or in combination with other radicals, denotes a —COOH group, and includes derivatives of carboxylic acid such as esters and amides. The term “ester” used herein, alone or in combination with other radicals, denotes —COO— group, and includes carboxylic acid derivatives, where the ester moieties are alkoxycarbonyl, such as methoxycarbonyl, ethoxycarbonyl, and the like, which may be substituted; aryloxycarbonyl group such as phenoxycarbonyl, napthyloxycarbonyl, and the like, which may be substituted, aralkoxycarbonyl group such as benzyloxycarbonyl, phenethyloxycarbonyl, napthylmethoxycarbonyl, and the like, which may be substituted; heteroaryloxycarbonyl, heteroaralkoxycarbonyl, wherein the heteroaryl group, is as defined above, which may be substituted; heterocyclyloxycarbonyl, where the heterocyclic group, as defined earlier, which may be substituted.

The term “amide” used herein, alone or in combination with other radicals, represents an aminocarbonyl radical (H₂N—C═O—), wherein the amino group is mono- or di-substituted or unsubstituted, such as methylamide, dimethylamide, ethylamide, diethylamide, and the like. The term “aminocarbonyl” used herein, either alone or in combination with other radicals, with other terms such as “aminocarbonylalkyl”, “N-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl”, and “N-alkyl-N-hydroxyaminocarbonylalkyl”, substituted or unsubstituted. The terms “N-alkylaminocabonyl” and “N,N-dialkylaminocarbonyl” denotes aminocarbonyl radicals, as defined above, which have been substituted with one alkyl radical and with two alkyl radicals, respectively. Preferred are “lower alkylaminocarbonyl” having lower alkyl radicals as described above attached to aminocarbonyl radical. The terms “N-arylaminocarbonyl” and “N-alkyl-N-arylammocarbonyl” denote aminocarbonyl radicals substituted, respectively, with one aryl radical, or one alkyl, and one aryl radical. The term “aminocarbonylalkyl” includes alkyl radicals substituted with aminocarbonyl radicals.

The term “hydroxyalkyl” used herein, either alone or in combination with other radicals, denotes an alkyl group, as defined above, substituted with one or more hydroxy radicals, such as hydroxymethyl, hydroxyethyl, hydroxypropyl, hydroxybutyl, hydroxypentyl, hydroxyhexyl and the like.

The term “aminoalkyl” used herein, alone or in combination with other radicals, denotes an amino (—NH₂) moiety attached to an alkyl radical, as defined above, which may be substituted, such as mono- and di-substituted aminoalkyl. The term “alkylamino” used herein, alone or in combination with other radicals, denotes an alkyl radical, as defined above, attached to an amino group, which may be substituted, such as mono- and di-substituted alkylamino.

The term “alkoxyalkyl” used herein, alone or in combination with other radicals, denotes an alkoxy group, as defined above, attached to an alkyl group, such as methoxymethyl, ethoxymethyl, methoxyethyl, ethoxyethyl and the like. The term “aryloxyalkyl” used herein, alone or in combination with other radicals, includes phenoxymethyl, napthyloxymethyl, and the like. The term “aralkoxyalkyl” used herein, alone or in combination with other radicals, includes C₆H₅CH₂OCH₂, C₆H₅CH₂OCH₂CH₂, and the like.

The term “alkylthio” used herein,. either alone or in combination with other radicals, denotes a straight or branched or cyclic monovalent substituent comprising an alkyl group of one to twelve carbon atoms, as defined above, linked through a divalent sulfur atom having a free valence bond from the sulfur atom, such as methylthio, ethylthio, propylthio, butylthio, pentylthio and the like. Examples of cyclic alkylthio are cyclopropylthio, cyclobutylthio, cyclopentylthio, cyclohexylthio and the like, which may be substituted.

The term “thioalkyl” used herein, either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be substituted.

The term “arylthio” used herein, either alone or in combination with other radicals, denotes an aryl group, as defined above, linked through a divalent sulfur atom, having a free valence bond from the sulfur atom such as phenylthio, napthylthio and the like.

The term “alkoxycarbonylamino” used herein, alone or in combination with other radicals, denotes an alkoxycarbonyl group, as defined above, attached to an amino group, such as methoxycarbonylamino, ethoxycarbonylamino, and the like. The term “aryloxycarbonylamino” used herein, alone or in combination with other radicals, denotes an aryloxycarbonyl group, as defined above, attached to the an amino group, such as C₆H₅OCONH, C₆H₅OCONCH₃, C₆H₅OCONC₂H₅, C₆H₄(CH₃O)CONH, C₆H₄(OCH₃)OCONH, and the like. The term “aralkoxycarbonylamino” used herein, alone or in combination with other radicals, denotes an aralkoxycarbonyl group, as defined above, attached to an amino group C₆H₅CH₂OCONH, C₆H₅CH₂CH₂CH₂OCONH, C₆H₅CH₂OCONHCH₃, C₆H₅CH₂OCONC₂H₅, C₆H₄(CH₃)CH₂OCONH, C₆H₄(OCH₃)CH₂OCONH, and the like.

The term “aminocarbonylamino”, “alkylamiocarbonylamino”, “dialkylaminocarbonylamino” used herein, alone or in combination with other radicals, denotes a carbonylamino (—CONH₂) group, attached to amino(NH₂), alkylamino group or dialkylamino group respectively, where alkyl group is as defined above.

The term “amidino” used herein, either alone or in combination with other radicals, denotes a —C(═NH)—NH₂ radical. The term “alkylamidino” denotes an alkyl radical as discussed above, attached to an amidino group.

The tem “hydrazino” used herein, either alone or in combination with other radicals, denotes —NHNH—, suitably substituted with other radicals, such as alkyl hydrazino, where an alkyl group, as defined above is attached to a hydrazino group.

The term “alkoxyamino” used herein, alone or in combination with other radicals, denotes an alkoxy group, as defined above, attached to an amino group. The term “hydroxyamino” used herein, alone or in combination with other radicals, denotes —NHOH moiety, and may be substituted.

The term “sulfenyl” or “sulfenyl and its derivatives” used herein, alone or in combination with other radicals, denotes a bivalent group, —SO— or RₓSO, where Rₓ is substituted or unsubstituted alkyl aryl, heteroaryl, heterocyclyl, and the like.

The term “sulfonyl” or “sulfones and its derivatives” used herein, either alone or in combination with other radicals, with other terms such as alkylsulfonyl, denotes divalent radical —SO₂—, or RₓSO₂, where Rₓ is substituted or unsubstituted groups selected from alkyl aryl, heteroaryl, heterocyclyl, and the like. “Alkylsulfonyl” denotes alkyl radicals, as defined above, attached to a sulfonyl radical, such as methylsulfonyl, ethylsulfonyl, propylsulfonyl and the like.

The term “arylsulfonyl” used herein, either alone or in combination with other radicals, denotes aryl radicals, as defined above, attached to a sulfonyl radical, such as phenylsulfonyl and the like.

Suitable groups and substituents on the groups may be selected from those described anywhere in the specification.

Particularly useful compounds according to the present invention includes
2-3-Amino-3-{4-[2-(2,2-dimethyl-cyclopropyl)-quinoin-4-ylmethoxy]-phenyl}-2-oxo-pyrrolidin-1-yl)-4-methyl-pentanoic acid hydroxyamide;
2-{3-Amino-3-[4-2-methoxymethyl-quinolin-ylmethoxy)-phenyl]-2-oxo-pyrrolidin-1-yl}-4-methyl-pentanoic acid hydroxyamide;
2-{3-Amino-3-[4-(2-methoxymethylquinolin-4-ylmethoxy)-phenyl]-2-oxo-pyrrolidin-1-yl}-N-hydroxy-propionamnide;
N-Hydroxy-2-{3-[4-(2-methoxymethyl-quinolin-4-ylmethoxy)-phenyl]-3-methyl-2-oxo-pyrrolidin-1-yl}-propionamide;
2-{3-Amino-3-[4-2-isopropoxymethyl-quinolin-4-ylmethoxy)-phenyl]-2-oxo-pyrrolidin-1-yl}-4-methyl-pentanoic acid hydroxyamide;
2-{3-Amino-3-[4-(2-isopropoxymethyl-quinolin-4-ylmethoxy)-phenyl]-2-oxo-pyrrolidin-1 -yl}-N-hydroxy-propionamide;
2-(3-Amino-3-{4-[2-(2-methoxy-ethyl)quinolin-4-ylmethoxy]-phenyl}-2-oxo-pyrrolidin-1-yl)-4-methyl-pentanoic acid hydroxyamide;
2-{3-Amino-2-oxo-3-[4-(2-p-tolylsuinolin-4-ylmethoxy)-phenyl]-pyrrolidin-1-yl}-4-methyl-pentanoic acid hydroxyamide;
2-{3-Amino-2-oxo-3-[4-2-p-tolyl-quinolin-4-ylmethoxy)-phenyl]-pyrrolidin-1-yl}-N-hydroxy-propionamide;
2-(3-Amino-3-{4-[2-(4-chloro-phenyl)-quinolin-4-ylmethoxy]-phenyl)-2-oxo-pyrrolidin-1-yl}-4-methyl-pentanoic acid hydroxyamide;
2-[3-Amino-3-{4-[2-(4-chloro-phenyl)-quinolin-4-ylmethoxy]-phenyl]-2-oxo-pyrrolidin-1-yl}-N-hydroxy-propionamide;
2-3-Amino-3-{4-[2-(4-fluoro-phenyl)-quinohn-4-ylmethoxy]-phenyl}-2-oxo-pyrrolidin-1-yl)-4-methyl-pentanoic acid hydroxyamide;
2-(3-Amino-3-{4-[2-(4-fluoro-phenyl)-quinolin-4-ylmethoxy]-3-phenyl}-2-oxo-pyrrolidin-1-yl)-N-hydroxy-propionamide;
2-3-Amino-3-{4-[2-(4-methoxy)phenyl)-quinolin-4-ylmethoxy]-phenyl}-2-oxo-pyrrolidin-1-yl)-4-methyl-pentanoic acid hydroxyamide;
2-(3-Amino-3-{4-[2-(4-methoxy-phenyl)-quinolin-4-ylmethoxy]-phenyl}-2-oxo-pyrroldin-1-yl)-N-hydroxy-propionamide; N-Hydroxy-2-(3-{4-[2-4-methoxy-phenyl)-quinolin-4-ylmethoxy]-phenyl}-3-methyl-2-oxo-pyrrolidin-1-yl)-propionamide;
2-(3-Amino-3-{4-[2-(4-ethoxy-phenyl)-quinolin-4-ylmethoxy]-phenyl}-2-oxo-pyrrolidin-1-yl)-4-methyl-pentanoic acid hydroxyamide;
2-3-Amino-3-{4-[2-(4-ethoxy-phenyl)-quinolin-4-ylmethoxy]-phenyl}-2-oxo-pyrrolidin-1-yl)-N-hydroxy-propionamide;
2-(3-Amino-3-{4-[2-(4-benzyloxy-phenyl)-quinolin-4-ylmethoxy]-phenyl}-2-oxo-pyrrolidin-1-yl)-4-methyl-pentanoic acid hydroxyamide;
2-3-Amino-3-{4-[2-(4-benzyloxy-phenyl)-quinolin-4-ylmethoxy]-phenyl}-2-oxo-pyrrolidin-1-yl)-N-hydroxy-propionamide;
2-(3-Amino-3-{4-[2-(4-methylsulfanyl-phenyl)-quinolin-4-ylmethoxy]-phenyl}-2-oxo-pyrrolidin-1-yl)-4-methyl-pentanoic acid hydroxyamide;
2-3-Amino-3-{4-[2-(4-methylsulfanyl-phenyl)-quinolin-4-ylmethoxy]-phenyl}-2-oxo-pyrrolidin-1 -yl)-N-hydroxy-propionamide;
2-{3-Amino-2-oxo-3-[4-2-m-tolyl-quinolin-4-ylmethoxy)-phenyl]-pyrrolidin-1-yl}-4-methyl-pentanoic acid hydroxyamide;
2-{3-Amino-2-oxo-3-[4-(2-m-tolyl-quinolin-4-ylmethoxy)-phenyl]-pyrrolidin-1-yl}-N-hydroxy-propionamide;
2-(3-Amino-3-{4-[2-(3-chloro-phenyl)-quinolin-4-ylmethoxy]-phenyl}-2-oxo-pyrrolidin-1-yl)4-methyl-pentanoic acid hydroxyamide;
2-3-Amino-3-{4-[2-(3-chloro-phenyl)-quinolin-4-ylmethoxy]-phenyl}-2-oxo-pyrrolidin-1-yl)-N-hydroxy-propionamide;
2-(3-Amino-3-(4-[2-3-methoxphenyl)-quinolin-4-ylmethoxy]-phenyl)-2-oxo-pyrrolidin-1-yl)-4-methyl-pentanoic acid hydroxyamide;
2-(3-Amino-3-{4-[2-(3-methoxy-phenyl}-quinolin-4-ylmethoxy]-phenyl) -2-oxo-pyrrolidin-1-yl)-N-hydroxy-propionamide;
2-(3-Amino-3-{4-[2-5-chloro-thiophen-2-yl)-quinolin-4-ylmethoxy]-phenyl}-2-oxo-pyrrolidin-1-yl)-N-hydroxy-propionamide;
2-3-Amino-3-{4-[2-5-methyl-furan-2-yl)-quinolin-4-ylmethoxy]-phenyl}-2-oxo-pyrrolidin-1-yl)-4-methyl-pentanoic acid hydroxyamide;
N-Hydroxy-2-(3-methyl-3-{4-[2-(5-methyl-furan-2-yl)-quinolin-4-ylmethoxy]-phenyl}-2-oxo-pyrrolidin-1-yl)-propionamide;
2-(3-Amino-3-{4-[2-(5-methyl-furan-2-yl)-quinolin-4-ylmethoxy]-phenyl}-2-oxo-pyrrolidin-1-yl)-N-hydroxy-propionamide;
2-{3-Amino-3-[4-(4-methoxymethyl-benzyloxy)-phenyl]-2-oxo-pyrrolidin-1-yl}-4-methyl-pentanoic acid hydroxyamide;
2-{3-3-[4-2-methoxymethyl-quinolin-4-ylmethoxy)-phenyl]-2-oxo-pyrrolidin-1-yl}-4-methyl-pentanoic acid;
2-{3-Amino-3-[4-(2-isopropoxymethyl-quinolin-4-ylmethoxy)-phenyl]-2-oxo-pyrrolidin-1-yl}-4-methyl-pentanoic acid;
2-{3-Amino-2-oxo-3-[4-(2-p-tolyl-quinolin-4-ylmethoxy)-phenyl]-pyrrolidin-1-yl}4-methyl-pentanoic acid;
2-(3-Amino-3-{4-[2-(4-methoxy-phenyl}-quinolin-4-ylmethoxy]-phenyl)-2-oxo-pyrrolidin-1-yl)-propionic acid.

Several synthesis routes can be employed to prepare the compounds of the present invention well known to one skilled in the art of organic synthesis. The compounds of formula (1) can be synthesized using the methods described below, together with conventional techniques known to those skilled in the art of organic synthesis, or variations thereon as appreciated by those skilled in the art Referred methods include, but not limited to those described below.

When preparing or elaborating compounds of the invention containing heterocyclic rings, those skilled in the art recognize that substituents on the ring may be prepared before, after or concomitant with the construction of the pyrrolidinone ring. It is understood by those skilled in the art that the nature and order of the synthetic steps presented may be varied for the purpose of optimizing the formation of the compounds of the present invention. Those skilled in the art will recognize that certain reactions are carried out when other potentially reactive functionality on the molecule is masked or protected to avoid undesired side reactions and/or increasing the yield of the reaction. The desired protective groups may be found T. W. Greene, P. G. M. Wuts “Protective Groups in Organic Synthesis” 2^{nd} edition 1991, Wiley and Sons, New York. It is understood by one skilled in the art of organic synthesis that the protective groups present on various reactive functionality of the molecule must be compatible with reagents and reactions proposed and it will be readily apparent to one skilled in the art an alternate methods must be used. The reactions are performed in solvents appropriate to the reagents and materials used and are suitable for the transformations being effected. The pyrrolidinone compounds of the present invention may be prepared by methods outlined in schemes 1 & 2.

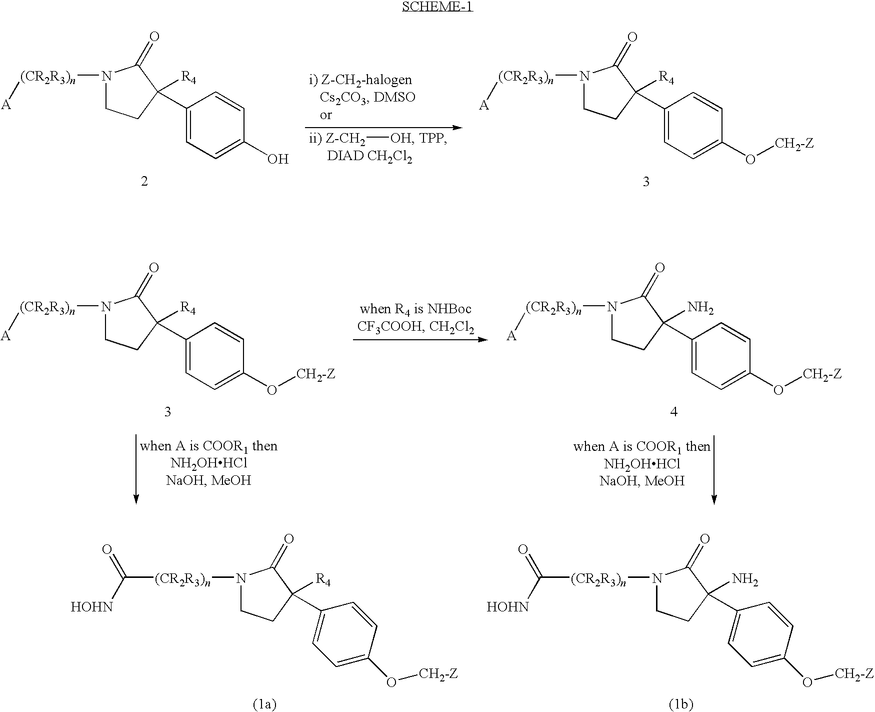

The compounds of the present invention may be prepared by treating suitable compound of formula (2) where A represent suitably substituted ester group, R₂, R₃ and R₄ are as defined earlier, with suitably substituted alkyl halide under basic condition using bases such as sodium carbonate, potassium carbonate, cesium carbonate, sodium hydroxide, potassium hydride, sodium hydride and the like or mixtures thereof, to give corresponding ether analogue (3) where R₂, R₃ & R₄ are as defined earlier and Y represents ‘OCH₂’. Compound of formula (3) represents compound of formula (I) where R₂, R₃ & R₄are as defined earlier and Y represents ‘OCH₂’. If the compound (3) is having suitable protective group, it is deprotected under acidic condition using trifluoroacetic acid in solvent like CH₂Cl₂, chloroform, dichloroethane and like or mixture thereof to get compound of formula (4). When compound (3) or (4) are ester, it is converted to its corresponding hydroxamic acid of formula (1a), where A represents hydroxamic acid and Y represents ‘OCH₂’ and (1b) respectively, where A represents hydroxamic acid, R₄ represents NH₂ and Y represents OCH₂ and all other symbols are as defined earlier by treatment with hydroxylamine under basic condition (KOK NaOH) in alcoholic solvent such as MeOH, EtOH and the like or mixtures thereof, while keeping the temperature of the reaction in the range of −20° C. to 50° C.

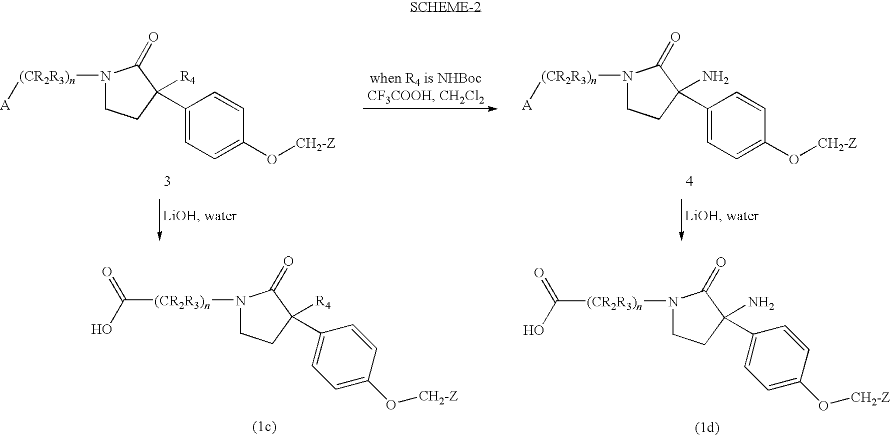

When compound (3) or (4) are suitably substituted ester, it is converted to its carboxylic acid of formula (1c) where A is COOH, Y is OCH₂ and other symbols are as defined earlier, and (1d) respectively where A represents hydroxamic acid, R₄ represents NH₂ and Y represents ‘OCH₂’ and all other symbols are as defined earlier by treatment with base such as KOK, NaOH, LiOH and like in water, THF, MeOH, EtOH and the like or mixtures thereof, while keeping the temperature of the reaction in the range of −10° C. to 50° C.

The compounds of the present invention may be formulated into suitable pharmaceutically acceptable salts by processes and using suitable agents as are known in the art.

Another aspect of the present invention comprises a pharmaceutical composition, containing at least one of the compounds of the general formula (I), their tautomeric forms, their pharmaceutically acceptable salts, as an active ingredient, together with pharmaceutically employed carriers diluent and the like.

The compounds of formula (I) or pharmaceutical compositions containing them may be administered either by conventional routes of administration.

Preferably the composition is in unit dosage form containing an effective amount of the active component, that is, the compounds of formula (I) according to this invention.

The quantity of active component, that is, the compounds of formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

In another aspect of the present invention, method of treatment and/or prevention of the diseases mentioned above are provided.

In a further aspect of the present invention, use of one or more compounds of the general formula (I) or pharmaceutically acceptable salts, for the preparation of a medicament thereof for the treatment and/or prevention of diseases mentioned in this document is provided.

The invention is explained in detail by the examples given below, which are provided by way of illustration only and therefore should not be construed to limit the scope of the invention.

1H MR spectral data given in the tables (vide infra) are recorded using a 300 M spectrometer (Bruker AVANCE-300) and reported in δ scale and the J values are in Hz. Until and otherwise mentioned the solvent used for NMR is DMSO-d₆ using Tetramethyl silane as the internal standard.

### EXAMPLE-1

### 2-{3-Amino-3-[4-2-methoxymethyl-quinolin-4-ylmethoxy)-phenyl]-2-oxo-pyrrolidin-1-yl}-4-methyl-pentanoic acid hydroxyamide (compound no. 2)

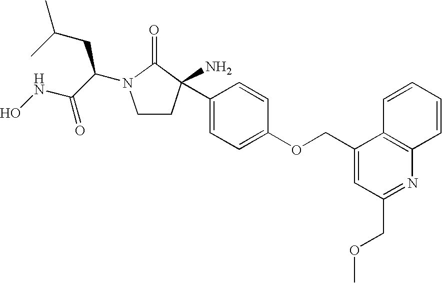 [0125] 1a) Diisopropyl azodicarboxylate (DIAD) (1.44 g, 7.12 mmol) was added to a solution of 2-[3-tert-butoxycarbonylamino-3-(4-hydroxy-phenyl)-2-oxo-pyrrolidin-1-yl]4-methyl-pentanoic acid methyl ester (2.0 g, 4.75 mmol), (2-methoxymethyl-quinolin-4-yl)-methanol (1.06 g, 5.23 mmol) and triphenyl phosphine (1.37 g, 5.23 mmol) in dichloromethane (20 mL) at 0° C. The mixture was stirred at 25-30° C. for 24 h. The reaction was quenched with 20 mL water and the organic layer was separated. The aq. layer was extracted with dichloromethane (20 mL). The combined organic layer was washed with water (20 mL) and dried over anhydrous magnesium sulfate (3 g). The solvent was distilled out under reduced pressure to get 2-{3-tert-butoxycarbonylamino-3-[4-(2-methoxymethyl-quinolin-4-ylmethoxy)-phenyl]-2-oxo-pyrrolidin-1-yl}-4-methyl-pentanoic acid methyl ester (1.8 g, yield 62.5%)
1b) Trifluoroacetic acid (3.38 g, 29.7 mmol) was added dropwise to a solution of 2-{3-tert-butoxycarbonylamino-3-[4-(2-methoxymethylquinolin-4-ylmethoxy)-phenyl]-2-oxo-pyrrolidin-1-yl}-4-methyl-pentanoic acid methyl ester (1.8 g, 2.97 mmol) obtained in (a) above in dichlorometiane (10 mL) at 0° C. The mixture was stirred at 25-30° C. for 4 h. Water (10 mL) was added and the pH of the mixture was adjusted to 10 by adding 10% aq. NaHCO₃ solution. The aqueous layer was extracted with dichloromethane (2×10 mL) and the combined organic layer was washed with water (10 mL). The solvent was distilled out under reduced pressure to get 2-{3-amino-3-[4-2-methoxymethyl-quinolin-4-ylmethoxy) -phenyl]-2-oxo-pyrrolidin-1-yl}-4-methyl-pentanoic acid methyl ester (1.4 g, yield 93.2%).
1c) A solution of sodium hydroxide (3.31 g, 55.2 mmol) in methanol (30 mL) was added to a hot solution of hydroxylamine hydrochloride (3.83 g, 55.2 mmol) in methanol (30 mL). The mixture was kept under stirring at 25-30° C. for 30 minutes and then cooled to 5-10° C. The precipitate was removed by filtration. This freshly prepared solution of the hydroxylamine was added to 2-{3-amino-3-[4-(2-methoxymethyl-quinolinylmethoxy)-phenyl]-2-oxo-pyrrolidin-1-yl}-4-methyl-pentanoic acid methyl ester (1.4 g, 2.76 mmol) obtained above in methanol (10 mL) at 5-10° C. The mixture was stirred at 25-30° C. for 2 h. and acidified to pH 6.0-6.5 with 1 N HCl. The hydroxamic acid was precipitated out. The desired 2-{3-amino-3-[4-(2-methoxymethyl-quinolin-4-ylmethoxy)-phenyl]-2-oxo-pyrrolidin-1 -yl}-4-methyl-pentanoic acid hydroxyamide was collected by filtration, washed with water (2×10 mL) and dried under vacuum to give the compound of formula (1) (1.0 g, 69.5%).

### EXAMPLE-2

### 2-(3-Amino-3-{4-[2-(4-methoxy-phenyl}-quinolin-4-ylmethoxy]-phenyl)-2-oxo-pyrrolidin-1-yl)-N-hydroxy-propionamide (compound no. 15)

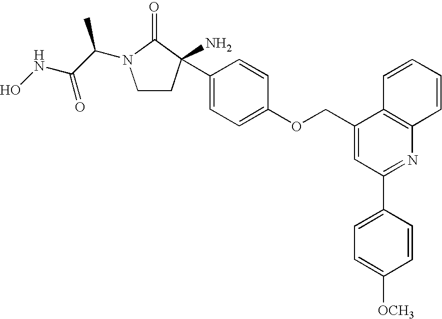 [0129] 2a) 2-[3-tert-butoxycarbonylamino-3-(4-hydroxy-phenyl)-2-oxo-pyrrolidin-1-yl]-propionic acid methyl ester (0.6 g, 1.42 mmol) was added to a suspension of 4chloromethyl-2-4-methoxy-phenyl)-quinoline (0.47 g, 1.5 mmol), cesium carbonate (1.4 g, 4.2 mmol) and sodium iodide (0.1 g, 0.7 mmol) in DMSO (6 mL). The mixture was bred at 25-30° C. for 4 h. ice water (30 mL) was added to the mixture and the precipitated solid was filtered off and dried. The crude compound was purified by column chromatography over silica gel (100-200 mesh, 15 % EtOAc in hexane) to get of 2-3-tert-butoxycarbonylamino-3-{4-[2-(4-methoxy-phenyl)-quinolin-4-ylmethoxy]-phenyl}-2-oxo-pyrrolidin-1-yl)-propionic acid methyl ester [0.6 g; yield 74%].
2b) Following a procedure analogous to (1b) above, 3-{4-[2(4-methoxy-phenyl)-quinolin-4-ylmethoxy]-phenyl}-2-oxo-pyrrolidin-1-yl-4-methyl-pentanoic acid methyl ester (0.55 g, 0.8 mmol) was deprotected with trifluoroacetic acid (1.26 mL,. 1.6 mol) in dicholromethane (5 mL) to get 2-3-Amino-3-{4-[2-4-methoxy-phenyl)-quinolin-4-ylmethoxy]-phenyl}-2-oxo-pyrrolidin-1-yl)-propionic acid methyl ester (0.38 g, yield 77%).
2c) Following a procedure analogous to (1c) above, the ester (0.30 g, 0.52 mmol) was reacted with hydroxylamine to give the titled hydroxamic acid (0.18 g, yield 60.1%).

### EXAMPLE-3

### Synthesis of 4-Chloromethyl-2-4-methoxy-phenyl)-quinoline.

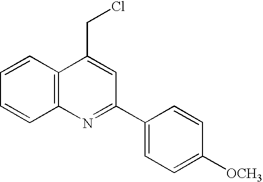 [0133] 3a) 2-(4-Methoxy-phenyl)-quinoline-4-carboxylic acid [J. Med. Chem., 40, 1794-1807 (1997)](6.0 g, 0.0215 mole) was suspended in SOCl₂ (18.5 mL, 0.2 mol) at 0° C. The mixture was refluxed for 2 h and cooled to 0° C. Methanol (50 mL) was added to get the hydrochloride salt of methyl ester derivative. Cold water (150 mL) was added to the mixture and its pH was adjusted to 7.0 with saturated with aq. NaHCO₃ solution. The precipitated solid was filtered, washed with cold water (2×10 mL) and dried under reduced pressure to get 2-(4-methoxy-phenyl)-quinoline-4-carboxylic acid methyl ester (5.0 g, yield 79.3%).
3b) Sodium borohydride (2.3 g, 0.06 mol) was added portionwise to a solution of 2-(4-methoxy-phenyl)-quinoline-4-carboxylic acid methyl ester (3.0 g) in TBF:water mixture (3:1, 12 mL) at 25-30° C. Mixture was stirred for 2 h at the same temperature. Cold water (30 mL) was added in to the mixture and pH was adjusted to 7.0 by adding acetic acid at 15-20° C. The resulting solid was filtered, washed with cold water (2×10 mL) and dried under reduced pressure to get [2-4-methoxy-phenyl)-quinolin-4-yl]-methanol (2.5 g, 92.6% )
3c) SOCl₂ (1.39 mL, 18.8 mmol) was added to a solution of [2-(4-methoxy-phenyl)-quinolin-4-yl]-methanol (2.0 g, 7.5 mmol) in dichloromethane (10 mL) at 0° C. The mixture was stirred at 25 to 30° C. for 2 h. The resulting yellow solid was filtered and washed with diisopropyl ether (2×10 mL) and the solid was dried under reduced pressure to get the desired title product as a hydrochloride salt (2.2 g, yield 95.7%).

A few of the compounds of the present invention prepared according to the similar procedure as described above, are listed below in table 1.

TABLE 1

| | |
|---|---|
| | I |
| 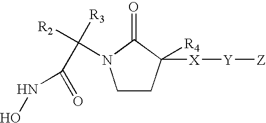 | |

| Compd. | Substituent in I | | | | | |
|---|---|---|---|---|---|---|
| No. | R₂ | R₃ | R₄ | X—Y—Z | | |
| 1. | H | i-Bu | NH₂ | 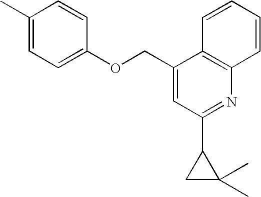 | Mol. Wt. = 530.66 | Yield = 62.5% |
| | 0.90 (9H, m), 1.02 (3H, d, J = 6.06), 1.51 (2H, t, J = 4.65), 1.62 (1H, m), 2.06-2.25 (4H, m), | | | | | |
| | 3.33-3.56 (4H, m), 4.5 (1H, m), 4.53 (1H, m), 5.57 (2H, s), 7.0 (2H, d, J = 8.58), 7.35 (1H, d, | | | | | |
| | J = 8.6), 7.43 (1H, d, J = 8.55), 7.53 (1H, t, J = 7.7), 7.6 (1H, s), 7.70 (1H, t, J = 7.5), 7.93 (1H, | | | | | |
| | d, J = 8.3), 8.06 (H, d, J = 8.25), 8.91 (1H, brs), 10.86 (1H, brs). | | | | | |
| 2. | H | i-Bu | NH₂ | 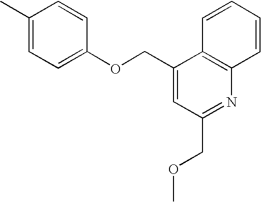 | Mol. Wt. = 506.59 | Yield = 33% |
| | 0.89 (3H, d, J = 5.79), 0.91 (3H, d, J = 6.03), 1.51 (2H, m), 1.68 (1H, m), 2.02 (2H, m), 2.08 | | | | | |
| | (2H, m), 3.37 (3H, s), 4.53 (1H, m), 4.63, (2H, s), 5.64 (2H, s), 7.06 (2H, d, J = 8.7), 7.34 | | | | | |
| | (2H, d, J = 8.67), 7.63 (1H, m), 7.75 (1H, m), 8.01 (1H, d, J = 8.25), 8.15 (1H, d, J = 8.25), | | | | | |
| | 8.91 (1H, brs), 10.87 (1H, brs). | | | | | |
| 3. | H | Me | NH₂ | 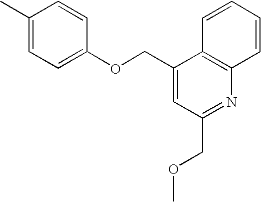 | Mol. Wt. = 464 | Yield = 22.9% |
| | 1.02 (3H, d, J = 6.06), 2.12 (2H, m), 3.36 (3H, s), 3.45-3.55 (2H, m), 4.5 (1H, m), 4.66 (2H, | | | | | |
| | s), 5.65 (2H, s), 7.05 (2H, d, J = 8.63), 7.39 (2H, d, J = 8.58), 7.63 (1H, m), 7.61-7.71 (2H, | | | | | |
| | m), 8.01 (1H, d, J = 8.43), 8.15 (1H, d, J = 8.04), 8.91 (1H, s), 10.73 (1H, s). | | | | | |
| 4. | H | Me | Me | 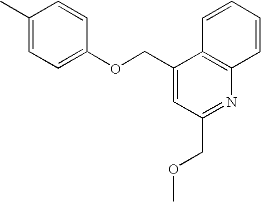 | Mol. Wt. = 463 | Yield = 48.9% |
| | 1.23-1.29 (6H, m), 2.26 (2H, m), 3.32 (3H, s), 3.40-3.50 (2H, m), 4.48 (1H, m), 4.67 (2H, | | | | | |
| | s), 5.65 (2H, s), 7.0 (2H, m), 7.39 (2H, m), 7.64 (1H, m), 7.75 (2H, m), 8.0 (1H, d, J = 8.37), | | | | | |
| | 8.15 (1H, d, J = 8.43), 8.84 (1H, d, J = 3.78), 10.71 (1H, brs). | | | | | |
| 5. | H | i-Bu | NH₂ | 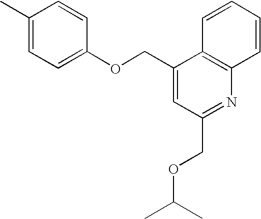 | Mol. Wt. = 534 | Yield = 15.6% |
| | 0.88 (3H, d, J = 5.94), 0.91 (3H, d, J = 6.12), 1.14 (6H, d, J = 6.06), 1.52 (1H, m), 2.05-2.10 | | | | | |
| | (4H, m), 3.38-3.47 (2H, m), 3.67 (1H, m), 4.53 (1H, m), 4.69 (2H, s), 5.64 (2H, s), 7.05 | | | | | |
| | (2H, d, J = 8.58), 7.34 (2H, d, J = 8.61), 7.62 (1H, t, J = 6.99), 7.73 (1H, m), 7.99 (1H, d, | | | | | |
| | J = 8.22), 8.12 (1H, d, J = 8.16), 8.90 (1H, s), 10.86 (1H, s). | | | | | |
| 6. | H | Me | NH₂ | 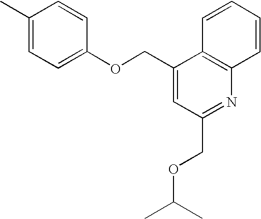 | Mol. Wt. = 492 | Yield = 39.6% |
| | 1.13 (6H, m) 1.31 (3H, d, J = 6.93), 2.26-2.36 (2H, m), 3.40-3.45 (2H, m), 3.68 (1H, m), | | | | | |
| | 4.52 (1H, m), 4.69 (2H, s), 5.69 (2H, s), 7.15 (3H, m), 7.40 (1H, t, J = 8.73), 7.63 (2H, t, | | | | | |
| | J = 7.53), 7.72 (1H, d, J = 4.0), 7.77 (1H, d, J = 7.44), 7.99 (1H, d, J = 8.1), 8.12 (1H, d, | | | | | |
| | J = 8.16), 8.94 (1H, d, J = 7.71), 10.77 (1H, s). | | | | | |
| 7. | H | i-Bu | NH₂ | 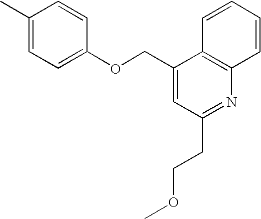 | Mol. Wt. = 520 | Yield = 66.6% |
| | 0.88 (3H, d, J = 5.82), 0.91 (3H, d, J = 6.03), 1.51 (1H, m), 2.05-2.08 (2H, m), 2.09-2.11 (2H, | | | | | |
| | m), 3.14 (2H, t, J = 6.54), 3.24 (3H, s), 3.37-3.40 (2H, m), 3.77 (2H, t, J = 6.57), 4.51 (1H, | | | | | |
| | m), 5.58 (2H, s), 7.0 (2H, d, J = 8.7), 7.4 (2H, m), 7.5 (2H, m), 7.7 (1H, m), 7.9 (2H, d, | | | | | |
| | J = 8.25), 8.0 (2H, d, J = 8.37), 8.91 (1H, s), 10.91 (1H, s), | | | | | |
| 8. | H | i-Bu | NH₂ | 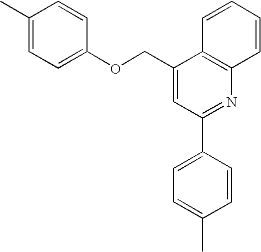 | Mol. Wt. = 552 | Yield = 34% |
| | 0.87 (3H, d, J = 5.76), 0.91 (3H, d, J = 6.15), 1.5 (1H, m), 2.05-2.09 (3H, m), 2.27 (1H, m), | | | | | |
| | 3.38-3.45 (2H, m), 4.5 (1H, m), 5.66 (2H, s), 7.09 (1H, d, J = 7.5), 7.36 (4H, m), 7.62 (1H, | | | | | |
| | t, J = 7.59), 8.09 (4H, m), 8.30 (1H, s), 8.90 (1H, s) 10.85 (1H, s). | | | | | |
| 9. | H | Me | NH₂ | 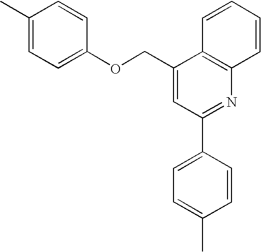 | Mol. Wt. = 510 | Yield = 76% |
| | 1.16 (3H, d, J = 7.11), 2.12 (2H, m), 2.16 (3H, s), 3.31-3.45 (2H, m), 4.51 (1H, m), 5.66 | | | | | |
| | (2H, s), 7.1 (2H, t, 3 = 7.26), 7.4 (4H, m), 7.62 (1H, t, J = 7.26), 7.79 (1H, t, J = 7.53), 8.10 | | | | | |
| | (1H, d, J = 9.3), 8.16 (3H, t, J = 6.81), 8.24 (1H, s), 8.91 (1H, s), 10.73 (1H, s). | | | | | |
| 10. | H | i-Bu | NH₂ | 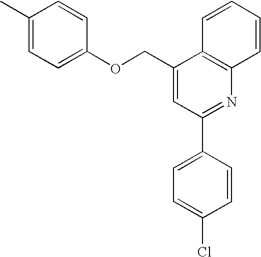 | Mol. Wt. = 573 | Yield = 77% |
| | 0.88 (3H, d, J = 5.85), 0.91 (3H, d, J = 6.03), 1.65 (1H, m), 2.05-2.20 (4H, m), 3.37-3.45 (2H, | | | | | |
| | m), 4.52 (1H, m), 5.67 (2H, s), 7.1 (1H, d, J = 8.67), 7.35 (2H, d, J = 8.64), 7.65 (3H, m), | | | | | |
| | 7.81 (1H, t, J = 7.29, 15.15), 8.12 (1H, d, J = 8.34), 8.20 (1H, d, J = 8.28), 8.30 (3H, m), 8.90 | | | | | |
| | (1H, s), 1058 (1H, s). | | | | | |
| 11. | H | Me | NH₂ | 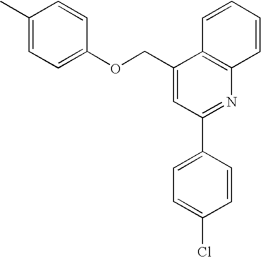 | Mol. Wt. = 531 | Yield = 38% |
| | 1.32 (3H, d, J = 7.77), 2.10 (2H, m), 3.32-3.45 (2H, m), 4.5 (1H, m), 5.67 (2H, s), 7.08 (2H, | | | | | |
| | m), 7.11 (2H, t, J = 7.02), 7.32 (1H, d, J = 8.67), 7.65 (3H, m), 7.82 (1H, t, J = 7.23), 8.12 (1H, | | | | | |
| | d, J = 8.46) 8.17 (1H, J = 3.7), 8.29 (3H, m), 8.91 (1H, s), 8.91 (1H, s), 10.63 (1H, s). | | | | | |
| 12. | H | i-Bu | NH₂ | 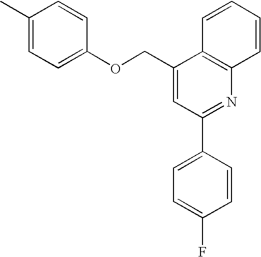 | Mol. Wt. = 556.6 | Yield = 56% |
| | 0.87 (3H, d, J = 5.82), 0.91 (3H, d, J = 6.00), 1.52 (1H, m), 2.08 (3H, m), 2.19 (1H, m), 3.38- | | | | | |
| | 3.45 (2H, m), 4.52 (1H, m), 5.67 (2H, s), 7.1 (1H, d, J = 8.73), 7.37 (4H, m), 7.64 (1H, t, J = | | | | | |
| | 7.44, 15.09), 7.81 (1H, t, J = 7.26, 15.24), 8.10 (1H, d, J = 8.43), 8.17 (1H, d, J = 8.10), | | | | | |
| | 8.32 (4H, s). 8.91 (1H, s), 10.86 (1H, s). | | | | | |
| 13. | H | Me | NH₂ | 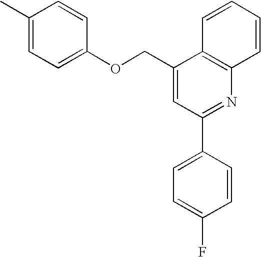 | Mol. Wt. = 514.55 | Yield = 52% |
| | 1.29 (3H, t, J = 6.9), 2.08 (2H, m), 3.31-3.41 (2H, m), 4.5 (1H, m), 5.67 (2H, s), 7.11 (2H, t, | | | | | |
| | J = 8.13), 7.32 (1H, d, J = 8.22), 7.39 (3H, t, J = 6.51), 7.64 (1H, t, J = 7.38), 7.81 (1H, t, | | | | | |
| | J = 7.29), 8.11 (1H, d, J = 8.37), 8.17 (1H, d, J = 8.19), 8.30(1H, s), 8.32 (2H, m), 10.73 (1H, | | | | | |
| | s). | | | | | |
| 14. | H | i-Bu | NH₂ | 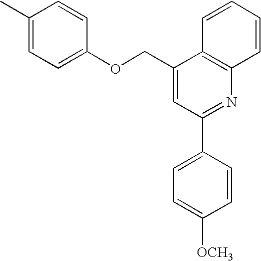 | Mol. Wt. = 568 | Yield = 60% |
| | 0.88 (3H, d, J = 5.91), 0.91 (3H, d, J = 5.97), 1.66 (1H, m), 2.10-2.19 (4H, m), 3.32-3.51 (2H, | | | | | |
| | m), 3.84 (3H, s), 4.51 (1H, m), 5.66 (2H, s), 7.12 (3H, t, J = 4.83), 7.37 (2H, d, J = 8.65), | | | | | |
| | 7.61 (1H, t, J = 7.56), 7.76 (1H, t, J = 7.23), 8.13 (1H, d, J = 11.2), 8.27 (1H, d, J = 7.47), 8.92 | | | | | |
| | (3H, J = 7.12), 10.1 (1H, s), 10.8 (1H, s). | | | | | |
| 15. | H | Me | NH₂ | 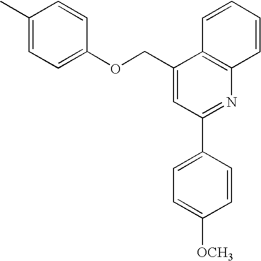 | Mol. Wt. = 526.5 | Yield = 86.2% |
| | 1.37 (3H, d, J = 7.23), 2.27-2.34 (2H, m), 3.30-3.48 (2H, m), 3.84 (3H, s), 4.65 (1H, m), | | | | | |
| | 5.68 (2H, s), 7.10 (4H, m), 7.32 (2H, d, J = 8.79), 7.58 (1H, d, J = 7.14), 7.76 (1H, d, J = 7.02), | | | | | |
| | 8.07 (1H, d, J = 8.15), 8.13 (1H, d, J = 8.17), 8.22 (3H, d, J = 8.72), 8.92 (s, 1H), 10.69 (s, | | | | | |
| | 1H). | | | | | |
| 16. | H | Me | Me | 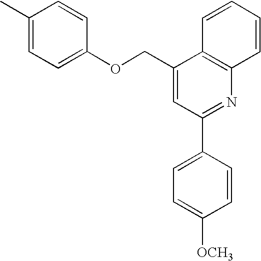 | Mol. Wt. = 525 | Yield = 51.1% |
| | 1.21 (3H, m), 1.27 (3H, s), 2.05 (1H, m), 2.23 (1H, m), 3.37 (2H, d, J = 7.35) 3.84 (3H, s) | | | | | |
| | 4.47 (1H, t, J = 6.96), 5.64 (2H, s), 7.10 (4H, d, J = 8.61), 7.28 (2H, t, J = 8.82), 7.60 (1H, t, | | | | | |
| | J = 7.47), 7.79 (1H, t, J = 7.23), 8.07 (2H, d, J = 8.40), 8.13 (3H, d, J = 8.22), 8.87 (1H, s), | | | | | |
| | 10.70 (1H, s). | | | | | |
| 17. | H | i-Bu | NH₂ | 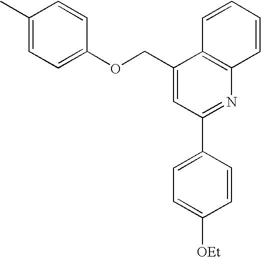 | Mol. Wt. = 582.69 | Yield = 51% |
| | 0.89 (3H, d, J = 5.85), 0.92 (3H, d, J = 6.00), 1.37 (3H, t, J = 6.93), 1.49 (1H, m), 2.09 (3H, | | | | | |
| | m), 2.13 (1H, m), 4.13 (1H, q, J = 6.66), 4.53 (1H, m), 5.66 (2H, s), 7.10 (4H, m), 7.35 (2H, | | | | | |
| | d, J = 8.64), 7.60 (1H, t, J = 7.44), 7.78 (1H, t, J = 7.23), 8.07 (1H, d, J = 8.43), 8.14 (1H, d, | | | | | |
| | J = 8.31), 8.22 (3H, m), 8.90 (1H, s), 10.86 (1H, s). | | | | | |
| 18. | H | Me | NH₂ | 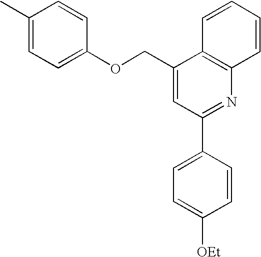 | Mol. Wt. = 540 | Yield = 30% |
| | 1.22 (3H, t, J = 6.81), 1.36 (3H, t, J = 6.96), 2.08 (1H, m), 2.24 (1H, m), 3.40-3.50, (2H, m), | | | | | |
| | 4.11 (2H, q, J = 9.96), 4.52 (1H, m), 5.64 (2H, s), 7.1 (4H, m), 7.32 (1H, d, J = 8.76), 7.38 | | | | | |
| | (1H, d, J = 8.76), 7.59 (1H, t, J = 7.5), 7.77 (1H, t, J = 7.41), 8.07 (1H, d, J = 8.43), 8.13 (1H, d, | | | | | |
| | J = 8.50), (1H, s,), 10.67 (1H, s). | | | | | |
| 19. | H | i-Bu | NH₂ | 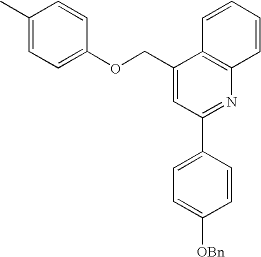 | Mol. Wt. = 644.76 | Yield = 57.1% |
| | 0.88 (3H, d, J = 5.70), 0.91 (3H, d, J = 5.76), 1.65 (1H, m), 2.05-2.26 (4H, m), 3.30-3.37 (2H, | | | | | |
| | m), 4.52 (1H, m), 5.20 (2H, s), 5.65 (2H, s), 7.09 (2H, d, J = 8.32), 7.18 (2H, d, J = 8.59), | | | | | |
| | 7.36 (3H, m), 7.41 (2H, t, J = 6.92), 7.49 (2H, d, J = 7.12), 7.59 (1H, t, J = 7.32), 7.77 (1H, t, | | | | | |
| | J = 7.0), 8.06 (1H, d, J = 8.34), 8.14 (1H, d, J = 8.21), 8.22 (3H, d, J = 8.12), 8.89 (1H, s), 10.84 | | | | | |
| | (1H, s). | | | | | |
| 20. | H | Me | NH₂ | 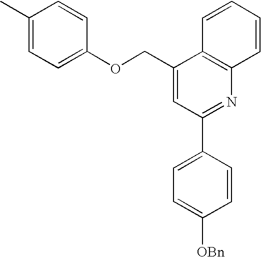 | Mol. Wt. = 602.76 | Yield = 93.4% |
| | 1.23 (3H, m), 2.06-2.26 (2H, m), 3.32-3.45 (2H, m), 4.51 (1H, m), 5.20 (2H, s), 5.65 (2H, | | | | | |
| | s), 7.11 (2H, t, J = 7.26), 7.19 (2H, dd, J = 8.50), 7.32 (2H, d, J = 8.39), 7.40 (3H, m), 7.48 | | | | | |
| | (2H, d, J = 6.84), 7.60 (1H, t, J = 7.15), 7.77 (1H, t, J = 7.26), 8.06 (1H, d, J = 8.22), 8.13 (1H, | | | | | |
| | d, J = 7.85), 8.23 (3H, d, J = 7.69), 8.93 (1H, s), 10.73 (1H, s). | | | | | |
| 21. | H | i-Bu | NH₂ | 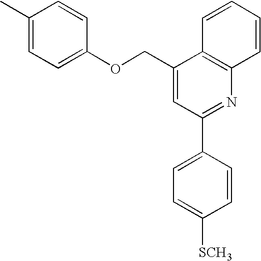 | Mol. Wt. = 584 | Yield = 43% |
| | 0.88 (3H, d, J = 5.73), 0.91 (3H, d, J = 5.85), 1.63 (1H, m), 2.10-2.35 (4H, m), 2.54 (3H, s), | | | | | |
| | 3.39-3.45 (2H, m), 4.52 (1H, m), 5.66 (2H, s), 7.10 (1H, d, J = 8.55), 7.34 (2H, d, J = 8.58), | | | | | |
| | 7.42 (1H, d, J = 8.31), 7.62 (1H, t, J = 7.38, 14.90), 7.79 (1H, t, J = 7.32), 8.09 (1H, d, | | | | | |
| | J = 8.49), 8.15 (1H, d, J = 8.37), 8.91 (1H, s), 10.86 (1H, s). | | | | | |
| 22. | H | Me | NH₂ | 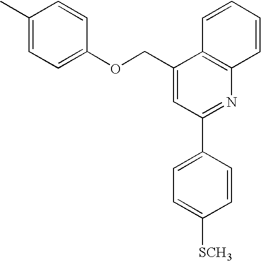 | Mol. Wt. = 542 | Yield = 31% |
| | 1.31 (3H, d, J = 6.0), 2.09 (1H, m), 2.24 (1H, m), 2.54 (3H, s), 3.40-3.50 (2H, m), 4.5 (1H, | | | | | |
| | m), 5.66 (2H, s), 7.11 (2H, d, J = 8.64), 7.32 (1H, d, J = 8.7), 7.41 (3H, t, J = 8.61), 7.62 (1H, t, | | | | | |
| | J = 7.35), 7.79 (1H, t, J = 7.32), 8.09 (1H, d, J = 8.25), 8.14 (1H, d, J = 8.46), 8.23 (3H, m), 8.90 | | | | | |
| | (1H, s), 10.73 (1H, s). | | | | | |
| 23. | H | i-Bu | NH₂ | 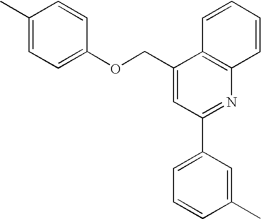 | Mol. Wt. = 552 | Yield = 88% |
| | 0.88 (3H, d, J = 5.61), 0.91 (3H, d, J = 5.91), 1.50 (2H, m), 1.63 (1H, m), 2.03 (2H, m), 2.43 | | | | | |
| | (3H, s), 3.32-3.41 (2H, m), 4.52 (1H, m), 5.66 (2H, s), 7.10 (2H, d, J = 8.46), 7.33 (3H, t, | | | | | |
| | J = 8.31), 7.44 (1H, t, J = 8.34), 7.63 (1H, t, J = 7.38), 7.80 (1H, t, J = 7.71), 8.02 (1H, d, | | | | | |
| | J = 7.59), 8.12 (3H, m), 8.25 (1H, s), 8.90 (1H, s), 10.85 (1H, s) | | | | | |
| 24. | H | Me | NH₂ | 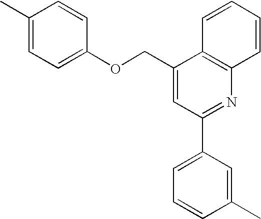 | Mol. Wt. = 510 | Yield = 87.5% |
| | 1.3 (3H, m), 2.07-2.16 (2H, m), 2.43 (3H, s), 3.31-3.45 (2H, m), 4.51 (1H, m), 5.6 (2H, s), | | | | | |
| | 7.12 (2H, t, J = 7.3), 7.32 (2H, d, J = 6.3), 7.44 (2H, q, J = 7.5), 7.64 (1H, t, J = 7.4), 7.80 (1H, | | | | | |
| | t, J = 7.1), 8.0 (1H, d, J = 7.55), 8.12 (3H, m), 8.25 (1H, s), 8.9 (1H, brs), 10.1 (1H, brs), | | | | | |
| | 10.73 (1H, brs). | | | | | |
| 25. | H | i-Bu | NH₂ | 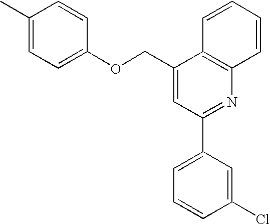 | Mol. Wt. = 573 | Yield = 67% |
| | 0.88 (3H, d, J = 5.83), 0.91 (3H, d, J = 6.03), 1.64 (1H, m), 2.08 (3H, m), 2.21 (1H, m), 3.48, | | | | | |
| | (2H, m), 4.50 (1H, m), 5.67 (2H, s), 7.10 (2H, d, J = 8.67), 7.34 (1H, d, J = 8.58), 7.59 (2H, | | | | | |
| | m), 7.69 (1H, t, J = 7.47), 7.82 (1H, t, J = 7.17), 8.18 (3H, m), 8.33 (2H, m), 8.90 (1H, s), | | | | | |
| | 10.85 (1H, s). | | | | | |
| 26. | H | Me | NH₂ | 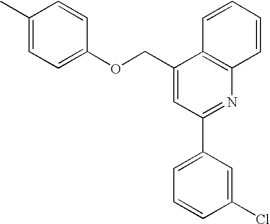 | Mol. Wt. = 531 | Yield = 71% |
| | 1.33 (3H, m), 2.11 (2H, m), 3.45-3.51 (2H, m), 4.5 (1H, m), 5.67 (2H, s), 7.11 (2H, t, | | | | | |
| | J = 8.31, 15.63), 7.32 (1H, d, J = 8.7), 7.42 (1H, d, J = 8.7), 7.59 (2H, m), 7.67 (1H, t, J = 7.5, | | | | | |
| | 14.88), 7.83 (1H, t, J = 7.32, 15.15), 8.18 (3H, m), 8.91 (2H, m), 8.90 (1H, s), 10.73 (1H, s). | | | | | |
| 27. | H | i-Bu | NH₂ | 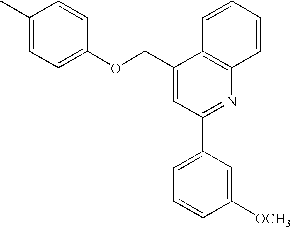 | Mol. Wt. = 568 | Yield 86.8% |
| | 0.87 (3H, d, J = 5.88), 0.91 (3H, d, J = 6.00), 1.64 (1H, m), 2.05-2.26 (4H, m), 3.39-3.45 (2H, | | | | | |
| | m), 3.87 (3H, s), 4.51 (1H, m), 5.6 (2H, s), 7.09 (3H, m), 7.34 (2H, d, J = 8.61), 7.47 (1H, t, | | | | | |
| | J = 8.07), 7.6 (1H, t, J = 7.47), 7.81 (3H, br d, J = 8.13), 8.12 (1H, d, J = 8.31), 8.17 (1H, d, | | | | | |
| | J = 8.4), 8.26 (1H, s), 10.0 (1H, s), 11.04 (1H, s). | | | | | |
| 28. | H | Me | NH₂ | 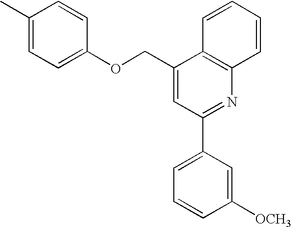 | Mol. Wt. = 526 | Yield = 25% |
| | 1.02 (3H, d, J = 6.06), 2.17-2.21 (2H, m), 3.32-3.48 (2H, m), 3.87 (3H, s), 4.51 (1H, m), | | | | | |
| | 5.69 (2H, s), 7.11 (3H, m), 7.35 (1H, d, J = 8.55), 7.43 (1H, t, J = 8.6), 7.49 (1H, d, J = 7.81), | | | | | |
| | 7.64 (1H, t, J = 7.25), 7.80 (3H, d, J = 9.33), 8.14 (1H, q, J = 8.4), 8.24 (1H, d, J = 3.1), 8.91 | | | | | |
| | (1H, s), 10.75 (1H, s) | | | | | |
| 29. | H | Me | NH₂ | 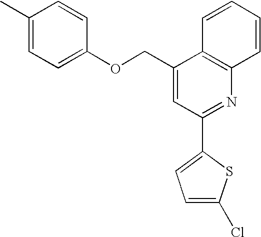 | Mol. Wt. = 536.5 | Yield = 43% |
| | 1.28 (3H, d, J = 6.26), 2.07 (2H, m), 3.32-3.45 (2H, m), 4.52 (1H, m), 5.61 (2H, s), 7.09 | | | | | |
| | (2H, m), 7.24 (1H, d, J = 3.91), 7.31 (1H, d, J = 8.49), 7.38 (1H, d, J = 8.54), 7.59 (1H, t, | | | | | |
| | J = 7.57), 7.75 (1H, t, J = 7.37), 7.89 (1H, s), 7.96 (1H, d, J = 8.37), 8.11 (1H, d, J = 8.07), 8.21, | | | | | |
| | (1H, s), 10.75 (1H, s). | | | | | |
| 30. | H | i-Bu | NH₂ | 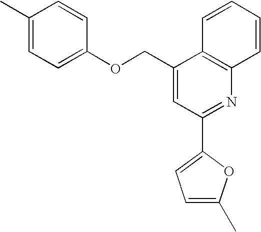 | Mol. Wt. = 542 | Yield = 66.6% |
| | 0.89 (3H, d, J = 5.82), 0.93 (3H, d, J = 6.03), 1.49-1.56 (2H, m), 1.63-1.66 (1H, m), 2.03-2.13 | | | | | |
| | (2H, m), 2.43 (3H, s), 3.32-3.38 (2H, m), 4.52 (1H, m), 5.63 (2H, s), 6.35 (1H, s), 7.09 | | | | | |
| | (2H, d, J = 8.52), 7.27 (1H, d), 7.36 (2H, d, J = 8.64), 7.59 (1H, t, J = 7.41), 7.71 (1H, t, | | | | | |
| | J = 7.29), 8.01 (2H, m), 8.12 (1H, d, J = 8.22), 8.92 (1H, s), 10.87 (1H, s). | | | | | |
| 31. | H | Me | Me | 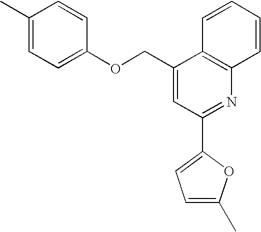 | Mol. Wt. = 499 | Yield = 69.56% |
| | 1.23 (3H, m), 1.38 (3H, s), 2.07 (1H, m), 2.11 (1H, m), 2.43 (3H, s) 3.45-3.55 (2H, m.78), | | | | | |
| | 4.50 (1H, t, J = 6.78), 5.63 (2H, s), 6.36 (1H, d, J = 2.73), 7.11 (2H, m) 7.32 (4H, m), 7.59 | | | | | |
| | (1H, t, J = 7.38), 7.77 (1H, t, J = 7.17), 8.01 (1H, m), 8.11 (1H, d, J = 8.34), 8.88 (1H, brs), | | | | | |
| | 10.72 (1H, brs). | | | | | |
| 32. | H | Me | NH₂ | 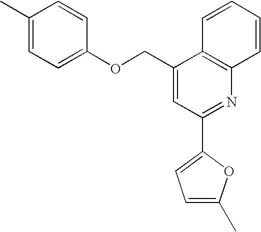 | Mol. Wt. = 500 | Yield = 56% |
| | 1.28 (3H, d, J = 5.68), 2.26 (2H, m), 2.42 (3H, s), 3.34-3.48 (2H, m), 4.51 (1H, m), 5.62 | | | | | |
| | (2H, s), 6.34 (1H, d), 7.10 (2H, t, J = 7.35), 7.24 (1H, d, J = 3.09), 7.33 (1H, d, J = 8.73), 7.40 | | | | | |
| | (1H, d, J = 8.7), 7.58 (1H, t, J = 7.23), 7.76 (1H, t, J = 7.14), 8.01 (2H, m), 8.10 (1H, d, J = 8.1), | | | | | |
| | 10.73 (1H, s). | | | | | |
| 33. | H | i-Bu | NH₂ | 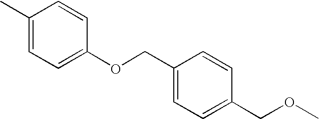 | Mol. Wt. = 465 | Yield = 89.6% |

0.87 (3H, d, J = 5.85), 0.91 (3H, d, J = 6.00), 1.51 (1H, m), 2.09 (3H, m), 2.09 (3H, m), 2.27
(1H, m), 3.14 (3H, s), 3.39-3.45 (2H, m), 4.39 (2H, s), 4.51 (1H, m), 5.06 (2H, s), 6.89
(2H, d, J = 8.61), 7.13 (4H, m) 7.40 (2H, d, J = 7.92), 8.91 (1H, s), 10.98 (1H, brs).

### EXAMPLE-4

### 2-{3-Amino-3-[4-(2-methoxymethyl-quinolin-4-ylmethoxy)-phenyl]-2-oxo-pyrrolidin-1-yl}-4-methyl-pentanoic acid (compound no. 34)

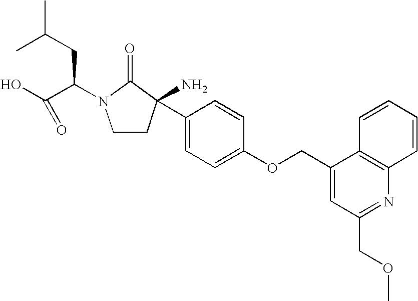

The compound obtained in (1b) in Example 1 above (1.0 g, 1.65 mmol) was suspended in a mixture of water (5 mL) and THF (5 mL) and a solution of LiOH (0.2 g, 4.95 mmol) in water (5 mL) was added. The mire was sired for 3 h at 25-30° C. The pH of the m was adjusted to 6.0 by adding dil. HCL. The compound was extracted in EtOAc (2×20 mL). The combined organic layer was washed with water (2×20 mL) and the solvent was distilled out under reduced pressure to get 2-{3-tert-butoxycarbonylamino-3-[4-(2-methoxymethyl-quinolin-4-ylmethoxy)-phenyl]-2-oxo-pyrrolidin-1-yl}4-methyl-pentamoic acid (0.8 g, yield 82%).
A few of the compounds of the present invention prepared according to the similar procedure as described in example 3 above, are listed below in table 2.

TABLE 2

| Compd. | Substituent in I | | | | | |
|---|---|---|---|---|---|---|
| No. | R₂ | R₃ | R₄ | X—Y—Z | | |
| 34. | H | i-Bu | NH₂ | 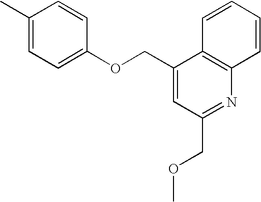 | Mol .Wt. = 491.58 | Yield = 72.4% |
| | 0.88 (3H, d, J = 6.14), 0.91 (3H, d, J = 6.31), 1.72 (1H, m), 2.09-2.26 (4H, m), 3.34 (3H, s), | | | | | |
| | 3.40-3.51 (2H, m), 4.60 (1H, m), 4.66 (2H, s), 5.64 (2H, s), 7.0 (2H, d, J = 8.63), 7.4 | | | | | |
| | (2H, d, J = 8.50), 7.6 (2H, m), 7.7 (1H, m), 7.9 (1H, d, J = 8.34), 8.1 (1H, d, J = 8.34). | | | | | |
| 35. | H | i-Bu | NH₂ | 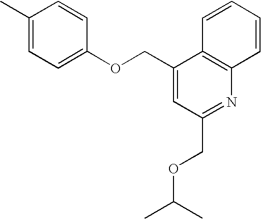 | Mol. Wt. = 519 | Yield = 6.88% |
| | 0.88 (3H, d, J = 5.94), 0.93 (3H, d, J = 6.33), 1.15 (6H, d, J = 6.06), 1.65 (1H, m), 2.11-2.17 | | | | | |
| | (4H, m), 3.40-3.49 (2H, m), 3.62 (1H, m), 4.60 (1H, m), 4.70 (2H, s), 5.66 (2H, s), 7.04 | | | | | |
| | (2H, d, J = 8.73), 7.43 (2H, d, J = 8.67), 7.63 (1H, t, J = 7.83), 7.74 (1H, s), 7.78 (1H, d, | | | | | |
| | J = 7.89), 8.00 (1H, J = 8.22), 8.13 (1H, d, J = 8.07). | | | | | |
| 36. | H | i-Bu | NH₂ | 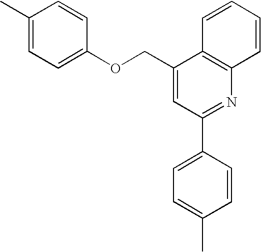 | Mol. Wt = 552.66 | Yield = 56.8% |
| | 0.87 (3H, d, J = 6.33), 0.91 (3H, d, J = 6.46), 1.48 (1H, m), 2.06 (2H, m), 2.26 (2H, m), 2.39 | | | | | |
| | (3H, s), 3.32-3.45 (2H, m), 4.53 (1H, m), 5.65 (2H, s), 7.09 (2H, d, J = 8.70), 7.36 (2H, d, J = | | | | | |
| | 8.01), 7.49 (2H, d, J = 8.58), 7.61 (1H, t, J = 7.17), 7.78 (1H, t, J = 6.96), 8.09 (1H, d, J = | | | | | |
| | 8.09), 8.15 (3H, m), 8.24 (1H, s), 8.09 (1H, d, J = 8.09), 8.15 (3H, m), 8.24 (1H, s). | | | | | |
| 37. | H | Me | NH₂ | 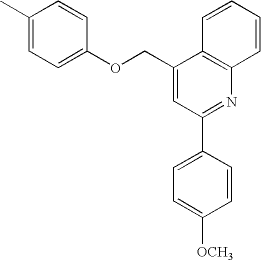 | Mol. Wt. = 511.6 | Yield = 68.75% |

1.37 (3H, d, J = 7.30), 2.27-2.34 (2H, m), 3.30-3.48 (2H, m), 3.84 (3H, s), 4.65 (1H, m),
5.68 (2H, s), 7.11 (2H, d, J = 8.71), 7.20 (2H, t, J = 8.63), 7.46 (2H, q, J = 8.69), 7.60 (1H, t,
J = 7.49), 7.78 (1H, t, J = 7.23), 8.07 (1H, d, J = 8.34), 8.07 (1H, d, J = 8.34), 8.13 (1H, d, J =
8.22), 8.23 (1H, d, J = 7.08).

### In Vitro Screening for TNF-α Inhibitory Activity Using Rat Whole Blood Assay:

Rats were deeply anaesthetized with anesthetic ether and blood (6-8 mL) was collected through retro-orbital plexus in a tube containing heparin (100 IU/mL). Five hundred microlitre blood sample was incubated with different concentrations of the test substances for 15 min at 37° C. LPS (1 μg/mL, final concentration) was subsequently added and the mixture was further incubated for 5 h at 37° C. The reaction was terminated by placing the samples over ice for 15 min. The samples were then centrifuged at 1000 g for 15 min at 4° C. The plasma was separated and stored in deep freezer until the assay. TNF-α levels were estimated in the treated plasma samples by ELISA method using commercially available kit (R&D System, Inc., USA). The inhibition of the TNF-α□□ production may be taken as an indicator of inhibition of TACE activity.

| | | % of TNF-α inhibition |
|---|---|---|
| Sl. No. | Compound No. | at 10 μM dose |
| 1. | 2 | 92 |
| 2. | 5 | 94 |
| 3. | 7 | 85 |
| 4. | 8 | 84 |
| 5. | 9 | 82 |
| 6. | 14 | 85 |
| 7. | 15 | 84 |
| 8. | 27 | 79 |

### In Vivo Screening for TNF-α Inhibitory Activity Using Wistar Rats:

After grouping based on body weights, the rats were kept in the experimental room for acclimatization for at least a day before the experiment On the day of experiment, rats were weighed again and administered with the test compounds at the specified dose (calculated as mg/kg on body weight basis) by oral gavage. The control group received vehicle alone. Thirty minutes after the administration of the test compound, rats were given an intravenous LPS (Sigma, 100 μg/kg) challenge. Ninety minutes after the LPS injection 0.5 mL blood was collected from retro-orbital plexus in heparin (10 IU/mL) coning tubes. Blood was centrifuged at 10000 rpm for 5 minute at 4° C. The plasma was separated and stored in deep freezer until the assay. TNF-α levels were estimated in the treated plasma samples by ELISA method using commercially available kit (R&D System, Inc., USA). The inhibition of the TNF-α □ production may be taken as an indicator of inhibition of TACE activity

### Uses:

The compounds of formula 1 possess matrix metalloprotease (MMP) and/or aggrecanase and/or TNF-α inhibitory activity. The compounds of the present invention are therefore useful for the treatment of diseases associated with MMP, aggrecanase and TNF-α.

1. (canceled)

2. A compound as claimed in claim 12 where Z is selected from pyrrolidinyl, imidazolidinyl, piperidinyl, piperazinyl, dihydrothiophene, dihydropyran, dihydrofuran, dihydrothiazole, pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzopyranyl, benzopyranonyl, benzofuranyl, benzothienyl, indolinyl, indolyl, azaindolyl, azaindolinyl, benzodihydrofuranyl, benzodihydrothienyl, pyrazolopyrimidinyl, pyrazolopyrimidonyl, azaquinazolinyl, azaquinazolinoyl, pyridofuranyl, pyridothienyl, thienopyrimidyl, thienopyrimidonyl, quinolinyl, pyrimidinyl, pyrazolyl, quinazolinyl, quinazolonyl, pyrimidonyl, pyridazinyl, triazinyl, benzoxazinyl, benzoxazinonyl, benzothiazinyl, benzothiazinonyl, benzoxazolyl, benzothiazolyl, benzimidazolyl, benzotriazolyl group; more preferably Z represents quinolinyl, pyrimidinyl, quinazolinyl groups.

3. A compound of claim 12 wherein the substitutions on any substituent as claimed in claim 12 may be selected from hydroxyl, oxo, halo, thio, nitro, amino, cyano, formyl, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocyclyl, heteroaryl, heterocycloalkyl, heteroaralkyl, heteroaryloxy, heteroaralkoxy, heterocyclyloxy, heterocyclylalkoxy, heterocyclylalkoxyacyl, acyl, acyloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, arylthio, alkylsulfonylamino, aminocarbonylamino, alkylaminocarbonylamino, alkoxyamino, hydroxyl amino, sulfonyloxy, alkylsulfonyloxy, alkoxycarbonylamino, aryloxycarbonylamino, aralkyloxycarbonylamino sulfenyl derivatives, sulfonyl derivatives, sulfonic acid and its derivatives.

4. A compound of claim 12 selected from
2-(3-Amino-3-{4-[2-(2,2-dimethyl-cyclopropyl)-quinolin-4-ylmethoxy]-phenyl}-2-oxo-pyrrolidin-1-yl)-4-methyl-pentanoic acid hydroxyamide;
2-{3-Amino-3-[4-(2-methoxymethyl-quinolin-4-ylmethoxy)-phenyl]-2-oxo-pyrrolidin-1-yl}-4-methyl-pentanoic acid hydroxyamide;
2-{3-Amino-3-[4-(2-methoxymethyl-quinolin-4-ylmethoxy)-phenyl]-2-oxo-pyrrolidin-1-yl}-N-hydroxy-propionamide;
N-Hydroxy-2-{3-[4-(2-methoxymethyl-quinolin-4-ylmethoxy)-phenyl]-3-methyl-2-oxo-pyrrolidin-1-yl}-propionamide;
2-{3-Amino-3-[4-(2-isopropoxymethyl-quinolin-4-ylmethoxy)-phenyl]-2-oxo-pyrrolidin-1-yl}-4-methyl-pentanoic acid hydroxyamide;
2-{3-Amino-3-[4-(2-isopropoxymethyl-quinolin-4-ylmethoxy)-phenyl]-2-oxo-pyrrolidin-1-yl}-N-hydroxy-propionamide;
2-(3-Amino-3-{4-[2-(2-methoxy-ethyl)-quinolin-4-ylmethoxy]-phenyl}-2-oxo-pyrrolidin-1-yl)-4-methyl-pentanoic acid hydroxyamide;
2-{3-Amino-2-oxo-3-[4-(2-p-tolyl-quinolin-4-ylmethoxy)-phenyl]-pyrrolidin-1-yl}-4-methyl-pentanoic acid hydroxyamide;
2-{3-Amino-2-oxo-3-[4-(2-p-tolyl-quinolin-4-ylmethoxy)-phenyl]-pyrrolidin-1-yl}-N-hydroxy-propionamide;
2-(3-Amino-3-{4-[2-(4-chloro-phenyl}-quinolin-4-ylmethoxy]-phenyl)-2-oxo-pyrrolidin-1-yl)-4-methyl-pentanoic acid hydroxyamide;
2-(3-Amino-3-{4-[2-(4-chloro-phenyl}-quinolin-4-ylmethoxy]-phenyl)-2-oxo-pyrrolidin-1-yl)-N-hydroxy-propionamide;
2-(3-Amino-3-{4-[2-(4-fluoro-phenyl)-quinolin-4-ylmethoxy]-phenyl}-2-oxo-pyrrolidin-1-yl)-4-methyl-pentanoic acid hydroxyamide;
2-(3-Amino-3-{4-[2-(4-fluoro-phenyl)-quinolin-4-ylmethoxy]-phenyl}-2-oxo-pyrrolidin-1-yl)-N-hydroxy-propionamide;
2-(3-Amino-3-{4-[2-(4-methoxy-phenyl)-quinolin-4-ylmethoxy]-phenyl}-2-oxo-pyrrolidin-1-yl)-4-methyl-pentanoic acid hydroxyamide;
2-(3-Amino-3-{4-[2-(4-methoxy-phenyl)-quinolin-4-ylmethoxy]-phenyl}-2-oxo-pyrrolidin-1-yl)-N-hydroxy-propionamide;
N-Hydroxy-2-(3-{4-[2-(4-methoxy-phenyl)-quinolin-4-ylmethoxy]-phenyl}-3-methyl-2-oxo-pyrrolidin-1-yl)-propionamide;
2-(3-Amino-3-{4-[2-(4-ethoxy-phenyl)-quinolin-4-ylmethoxy]-phenyl}-2-oxo-pyrrolidin-1 -yl)-4-methyl-pentanoic acid hydroxyamide;
2-(3-Amino-3-{4-[2-(4-ethoxy-phenyl)-quinolin-4-ylmethoxy]-phenyl}-2-oxo-pyrrolidin-1-yl)-N-hydroxy-propionamide;
2-(3-Amino-3-{4-[2-(4-benzyloxy-phenyl)-quinolin-4-ylmethoxy]-phenyl}-2-oxo-pyrrolidin-1-yl)-4-methyl-pentanoic acid hydroxyamide;
2-(3-Amino-3-{4-[2-(4-benzyloxy-phenyl)-quinolin-4-ylmethoxy]-phenyl}-2-oxo-pyrrolidin-1-yl)-N-hydroxy-propionamide;
2-(3-Amino-3-{4-[2-(4-methylsulfanyl-phenyl)-quinolin-4-ylmethoxy]-phenyl}-2-oxo-pyrrolidin-1-yl)-4-methyl-pentanoic acid hydroxyamide;
2-(3-Amino-3-{4-[2-(4-methylsulfanyl-phenyl)-quinolin-4-ylmethoxy]-phenyl}-2-oxo-pyrrolidin-1-yl)-N-hydroxy-propionamide;
2-{3-Amino-2-oxo-3-[4-(2-m-tolyl-quinolin-4-ylmethoxy)-phenyl]-pyrrolidin-1-yl}-4-methyl-pentanoic acid hydroxyamide;
2-{3-Amino-2-oxo-3-[4-(2-m-tolyl-quinolin-4-ylmethoxy)-phenyl]-pyrrolidin-1-yl}-N-hydroxy-propionamide;
2-(3-Amino-3-{4-[2-(3-chloro-phenyl)-quinolin-4-ylmethoxy]-phenyl}-2-oxo-pyrrolidin-1-yl)-4-methyl-pentanoic acid hydroxyamide;
2-(3-Amino-3-{4-[2-(3-chloro-phenyl)-quinolin-4-ylmethoxy]-phenyl}-2-oxo-pyrrolidin-1-yl)-N-hydroxy-propionamide;
2-(3-Amino-3-{4-[2-(3-methoxy-phenyl)-quinolin-4-ylmethoxy]-phenyl}-2-oxo-pyrrolidin-1-yl)-4-methyl-pentanoic acid hydroxyamide;
2-(3-Amino-3-{4-[2-(3-methoxy-phenyl)-quinolin-4-ylmethoxy]-phenyl}-2-oxo-pyrrolidin-1-yl)-N-hydroxy-propionamide;
2-(3-Amino-3-{4-[2-(5-chloro-thiophen-2-yl)-quinolin-4-ylmethoxy]-phenyl}-2-oxo-pyrrolidin-1-yl)-N-hydroxy-propionamide;
2-(3-Amino-3-{4-[2-(5-methyl-furan-2-yl)-quinolin-4-ylmethoxy]-phenyl}-2-oxo-pyrrolidin-1-yl)-4-methyl-pentanoic acid hydroxyamide;
N-Hydroxy-2-(3-methyl-3-{4-[2-(5-methyl-furan-2-yl)-quinolin-4-ylmethoxy]-phenyl}-2-oxo-pyrrolidin-1-yl)-propionamide;
2-(3-Amino-3-{4-[2-(5-methyl-furan-2-yl)-quinolin-4-ylmethoxy]-phenyl}-2-oxo-pyrrolidin-1-yl)-N-hydroxy-propionamide;
2-{3-Amino-3-[4-(4-methoxymethyl-benzyloxy)-phenyl]-2-oxo-pyrrolidin-1-yl}-4-methyl-pentanoic acid hydroxyamide;
2-{3-Amino-3-[4-(2-methoxymethyl-quinolin-4-ylmethoxy)-phenyl]-2-oxo-pyrrolidin-1-yl}-4-methyl-pentanoic acid;
2-{3-Amino-3-[4-(2-isopropoxymethyl-quinolin-4-ylmethoxy)-phenyl]-2-oxo-pyrrolidin-1-yl}-4-methyl-pentanoic acid;
2-{3-Amino-2-oxo-3-[4-(2-p-tolyl-quinolin-4-ylmethoxy)-phenyl]-pyrrolidin-1-yl}-4-methyl-pentanoic acid;
2-(3-Amino-3-{4-[2-[4-methoxy-phenyl)-quinolin-4-ylmethoxy]-phenyl]-2-oxo-pyrrolidin-1-yl}-propionic acid.

5. A pharmaceutical composition, which comprises a compound as defined in claim 12, and a pharmaceutically acceptable carrier, diluents or excipients or solvate.

6. A pharmaceutical composition according to claim 5, in the form of tablets, pills, capsules, powder, granules, syrup, solution or suspension.

7. A method for inhibition of production or action of TACE, MMP's and aggrecanase comprising administering a therapeutically acceptable amount of compound of formula (I) as claimed in claim 12, or a therapeutically acceptable salt or prodrug thereof.

8. A method of treatment or prophylaxis of various inflammatory, infectious, immununological or malignant diseases comprising administering an effective amount of a compound according to claim 12 to a mammal including human in need thereof.

9. Use of the compounds as claimed in claim 12 or their pharmaceutically acceptable salts for the preparation of medicine suitable for the treatment of diseases associated with excess of TNF-α (Tumour Necrosis Factor alpha) production or secretion.

10. A medicine for the treatment of diseases associated with excess of TNF-α (Tumour Necrosis Factor alpha) production or secretion comprising the compounds as claimed in claim 12, or their pharmaceutically acceptable salts.

11. A process for preparing compound of formula (I) as claimed in claim 12, comprising the steps of:
i) converting a compound of formula (2) to a compound of formula (3) where all the symbols are as defined in claim 12 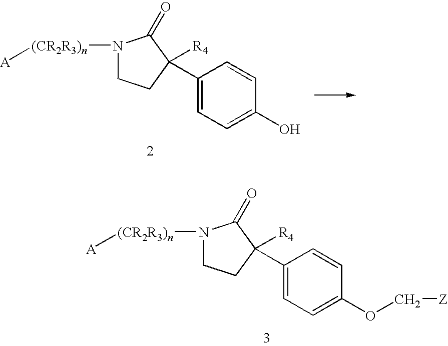
ii) covering a compound of formula (3) to compound of formula (1a), where all symbols are as defined in claim 12 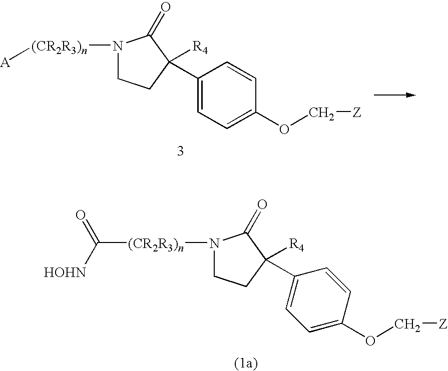
iii) optionally, converting a compound of formula (3) to a compound of formula (4) where all the symbols are as defined in claim 12 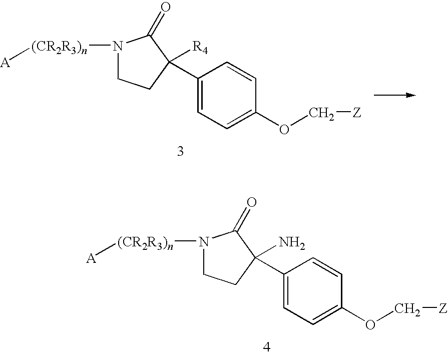
iv) converting the compound of formula (4) to further compound formula (1b), where all symbols are as defined earlier 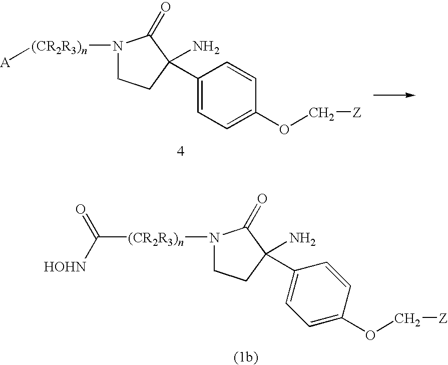
v) alternatively, compound of formula (3) may optionally be converted to compound of formula (1c), where all symbols are as defined earlier 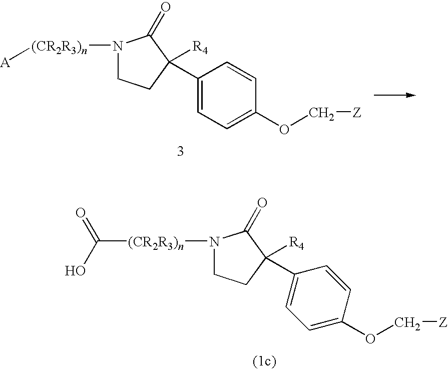
vi) alternatively, compound of formula (4) may optionally be converted to compound of formula (1d), where all symbols are as defined earlier

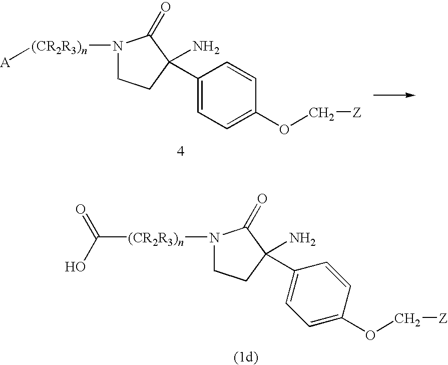

12. Compounds of the general formula (I), 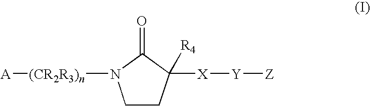
their stereoisomers, their tautomeric forms, their pharmaceutically acceptable salts, their pharmaceutically acceptable solvates, wherein
A is selected from CO₂H, CONHOH and CONHOR₁.
R₁ represents hydrogen, substituted or unsubstituted groups selected from linear or branched (C₁-C₈)alkyl; R₂ and R₃ may be same or different and independently represent hydrogen, substituted or unsubstituted linear or branched (C₁-C₈)alkyl, groups; X represents optionally substituted phenyl;
Z represents substituted phenyl, quinolinyl, pyrimidinyl groups;
The substituents on Z may be selected optionally from optionally substituted alkoxy alkyl or substituted groups selected from phenyl, or (5-6) membered heterocycle comprising 1-4 heteroatoms selected from the group consisting N, O and S; n=1;
Y represents (CR′R″)ₚ, O(CR′R″)ₚ, (CR′R″)ₚO, wherein p=0-2; R′ and R″ may be same or different and independently represent H, linear or branched substituted or unsubstituted (C₁-C₆)alkyl groups;
R₄ represents H, NR′R″, OR′, CN, (C₁-C₆)alkyl; R′ and R″ may be same or different and independently represent H, alkyl group, linear or branched substituted or unsubstituted (C₁-C₆)alkyl groups.
